(19) United States
(12) Patent Application Publication
(10) Pub. No.: US 2015/0068939 A1
(43) Pub. Date: Mar. 12, 2015

(54) MULTIFUNCTIONAL ENCLOSURE SYSTEM FOR MEDICAL PROBES AND METHOD OF USE

(71) Applicant: John Russell Seitz, III, Lincoln University, PA (US)

(72) Inventor: John Russell Seitz, III, Lincoln University, PA (US)

(21) Appl. No.: 14/217,051

(22) Filed: Mar. 17, 2014

Related U.S. Application Data
(63) Continuation-in-part of application No. 13/842,680, filed on Mar. 15, 2013.
Application No. 13/842,680 is a continuation-in-part of application No. 13/235,522, filed on Sep. 19, 2011.
(60) Provisional application No. 61/799,397, filed on Mar. 15, 2013.
(60) Provisional application No. 61/386,035, filed on Sep. 24, 2010.

(51) Int. Cl. A61B 19/02

(52) U.S. Cl. CPC A61B 19/026
USPC 206/363

(57) ABSTRACT

A multifunctional enclosure for medical probes and particularly an elongated probe member such as endoscope probe is described. The multifunctional enclosure provides an elongated sleeve that is configured to extend over a medical probe for the purpose of protecting the probe during transport and/or sterilization. An elongated sleeve may also provide a reservoir for pre-treating the medical probe with fluids or gels and may also provide a reservoir for quickly treating a medical probe with disinfectant or cleaning solution following a procedure. A multifunctional enclosure may comprise an handle housing that is configured to enclose at least a portion of a medical probe handle. An elongated sleeve may be detachable from a handle housing portion of a multifunctional enclosure and a liner may installed inside of the elongated sleeve.

This application claims the benefit of U.S. Provisional Patent Application No. 61/799,397 and U.S. patent Ser. No. 13/842,680 both filed on Mar. 15, 2013 and entitled MULTIFUNCTIONAL ENCLOSURE FOR MEDICAL PROBES AND METHOD OF USE; wherein U.S. patent Ser. No. 13/842,680 is a continuation-in-part of U.S. patent application Ser. No. 13/235,522 filed Sep. 19, 2011 and entitled MULTIFUNCTIONAL ENCLOSURE FOR MEDICAL PROBES AND METHOD OF USE, which claims the benefit of U.S. Provisional Patent Application No. 61/386,035, filed on Sep. 24, 2010, all of which are incorporated herein by reference in their entirety.

### BACKGROUND OF THE INVENTION

1. Field of the Invention

The invention relates to multifunctional enclosures for medical probes including endoscopes or instruments and methods of using the enclosure including but not limited to: protecting the medical probe during transport, storing and cleaning the medical probe, enclosing and protecting the medical probe during sterilization, contain hazardous post procedure infectious fluids and biomass and providing a post procedural reservoir for cleaning and/or sterilizing fluid.

2. Background

Medical probes are routinely used for various medical procedures whereby they are inserted into the body and in many cases are equipped with a means to view inside the body. Medical probes are expensive instruments that typically comprise an elongated member that is inserted into the body. These elongated members are often very delicate and designed to be flexible to allow for maneuvering and locating the tip at a desired location in the body. The flexibility and length of the elongated members of the medical probe make them difficult to handle. The elongated portions of the medical probe may unintentionally swing and hit objects during handling or transport and become damaged or contaminated.

Medical probes must be sterilized or disinfected after each use, which requires a considerable amount of handling including transport to a cleaning facility, transport to and handling during sterilization/disinfection, transport to and handling during storage, and finally transport to and handling during medical procedures. After a medical procedure is complete, the contaminated medical probe may pose a contamination risk to the operating room, and other areas exposed to a used probe as well as to patients and medical staff. Body fluids and other body contaminates may drip or fling off of the elongated member of the medical probe, and create a cross contamination risk. Fluids and biomaterials not immediately removed or soaked in disinfectant can become hardened to a medical probe and become a significant cross contamination risk.

During sterilization/disinfection procedures, the medical probe, or in some cases the elongated member of the medical probe, may be placed into a sterilization/disinfection chamber, such as, steam, ethylene oxide, hydrogen peroxide, ozone or the like. Handling of the medical probe during these repetitive procedures introduces possible cross contamination by placing the device directly on surfaces where other devices have previously been located. In addition, these repetitive procedures subject the medical probe to damage, along the length, and especially to the highly sensitive tip of the medical probe.

There exists a need for a multifunctional enclosure for medical probes that can be used to protect the elongated member of the medical probe during transport, storage, cleaning, sterilization, and provide a post procedural reservoir for cleaning and/or sterilizing fluid. There exists a need for quickly determining if the medical probe is clean and ready for a procedure or dirty and in need of cleaning and/or sterilization. There exists a need for a transport device that can be used to efficiently and effectively transport one or more medical probes whereby they are adequately protected from damage and contamination.

### SUMMARY OF THE INVENTION

The invention is directed to a multifunctional enclosure for medical probes and in particular the elongated members of an endoscopic device and a transport cart for the multifunctional enclosures. The multifunctional enclosure comprises an elongated sleeve having a first end, a second end and in some embodiments an attachment component. The first end may be dosed and the elongated member of the medical probe may be inserted through the second end. The elongated sleeve may be rigid in order to prevent the elongated member of the medical probe from swinging or moving during transport or handling. In one embodiment, the elongated sleeve is a tube, and may be a plastic tube. The elongated sleeve may be configured in a generally straight shape, or comprise at least one bend or curve. The elongated sleeve or a portion thereof may have a funnel shape, whereby the inner diameter is reduced over the length of the elongated member. A funnel shape elongated sleeve that is reduced in diameter toward the first end may minimize the amount of movement of a tip of a medical probe. In one embodiment, the elongated sleeve telescopes from the second end to the first end. In addition, the elongated sleeve as described herein, may be a “U” shape, or configured in a helical shape, wherein at least a portion of the elongated sleeve is in a helical configuration. The elongated sleeve may have an aspect ratio of greater than about three, greater than about five, greater than about ten, greater than about twenty, greater than about fifty and any range between an including the aspect ratios listed. An elongated sleeve or portion thereof may be telescoping, whereby one portion of the elongated sleeve may be stored within an adjacent portion of the elongated sleeve. A telescoping elongated sleeve may comprises a plurality of discrete sleeve portions that are coupled together and nested one within another. A telescoping elongated sleeve may be configured to extend from a first un-extended length to a second extended length and lock at the second extended length. A first telescoping portion may have a groove or pocket whereby a protrusion on a second telescoping portion may nest to lock the first telescoping portion with the second telescoping portion.

The first end of the multifunctional enclosure may be dosed or may comprise a detachable first end cover portion, such as a cap or plug. The first end cover portion may be removed during cleaning or sterilization procedures to allow for flow through the elongated sleeve. In addition, the first end cover portion may be removed to allow for adequate drying after cleaning or sterilization. In one embodiment the first end, or first end cover portion comprises a vent. A vent may be configured anywhere on the multifunctional enclosure, and the vent may be antimicrobial, and/or hydrophobic. In yet another embodiment the first end cover portion comprises a seam where the first end of the multifunctional enclosure has been sealed. For example, the first end of a plastic multifunctional enclosure may be heat pressed and welded together for form a seal. In still another embodiment, the first end of the multifunctional enclosure may be molded in such a way to form a cover over the end of the elongated member of the multifunctional enclosure. For example the elongated sleeve may be blow molded whereby the elongated sleeve is a contiguous piece of material and the first end is molded therein.

A pouch, cap or plug may be attached or sealed over the first end or second end to protect interior of the multifunctional enclosure from contaminates prior to use. The pouch, cap or plug may be removed prior to use and a clean first end cover portion may be used attached to the first end. The first end may also comprise a protective feature, such as a foam covering over at least a portion of the first end and extending up from the first end of the elongated sleeve. A protective feature may be configured over the first end and/or along the elongated sleeve near the first end. A protective cover may be a foam sleeve that can be slid onto or over the first end of the elongated sleeve whereby the protective feature can be removed prior to sterilization or after transport from on location to another.

The multifunctional enclosure as describe herein may further comprise a probe tip retainer. In one embodiment, a probe tip retainer is an additional retaining element that is configured within the elongated sleeve and retains the probe within the elongated sleeve. A probe tip retainer may comprise a ring of material that is configured within the elongated member and prevents or substantially reduces the movement of the probe tip within the elongated member, and in some case may prevent the probe tip from hitting the inside walls of the elongated member when jostled. A probe tip retainer may be configured with a first end cover portion, and comprise a tip insert, wherein the probe tip may be located therein and substantially secured. A variety of probe tip retainers may be designed for the different types of medical probes. In yet another embodiment, a probe tip retainer is comprised of an indented portion, or reduced open area of the elongated member. For example, an elongated member may have a first interior diameter, and a portion of the elongated member, preferably proximate the first end, may have a reduced interior diameter, whereby the tip is more effectively restrained from movement with the elongated sleeve. An elongated sleeve may comprise two or more indented portions to act as probe tip retainers along the length of the elongated sleeve.

The second end of the multifunctional enclosure may comprise an enlarged end or opening configured to facilitate the insertion of the medical probe. The second end may be a funnel shape or an irregular shape. In one embodiment, the second end comprises a cover that may be removed to allow for the insertion of the medical probe. In an alternative embodiment, the second end cover may comprise an opening, such as a hole or at least one slit, whereby the medical probe may be inserted. The second end cover may protect the interior of the multifunctional enclosure from contamination prior to use. In one embodiment the multifunctional enclosure, comprises two second end covers; one being solid, and the other having an opening. In this embodiment, the solid second end cover may be removed and the medical probe may be inserted through the opening in the other second end cover. The second end may further comprise at least one flute whereby the air may pass when a medical probe is inserted into the multifunctional enclosure. The flute or flutes may comprise channels along at least a portion of the length of the enlarged end. In one embodiment, the second end comprises a plurality of flutes extending the length of the enlarged end.

The attachment component may comprise any number of components configured to attach the multifunctional enclosure to the medical probe. The attachment component may include for example, elastic bands, hooks, latches, hook and loop fasteners and the like. In one embodiment, the attachment component comprises at least one elastic band that may be detachably attached to the medical probe, such as by stretching the band and locating the stretched end over a portion of the medical probe.

The multifunctional enclosure may further comprise an enclosure pouch that may be configured to enclose the end of the medical probe that extends from the multifunctional enclosure. In one embodiment, the enclosure pouch is attached to the multifunctional enclosure, and comprises a sealing portion along the extended end that may be used to seal the medical probe within the enclosure pouch. The second end of the multifunctional enclosure may comprise a handle housing that is configured to accept and protect at least a portion of a medical probe handle. A medical probe handle comprises the body of the medical probe that is manipulated by medical staff and may comprise controls, power connections and the like. A medical probe handle is typically much larger in dimension than the maximum cross-length dimension, ie diameter, of an elongated probe member. A handle housing may be configured to enclose a portion of a medical probe handle, or may be configured completely enclose a medical probe housing, or substantially enclose a medical probe housing, wherein power cords and other extensions may extend from the handle housing. Furthermore, a handle housing may be configured to be detached from an elongated sleeve of the present invention, or from a medical probe handle or both. In one embodiment, a handle housing is configured to open and may have a hinged element to allow the handle house to be opened and removed from the medical probe handle. In another embodiment, a handle housing comprises one or more pieces that may be snapped or otherwise attached to each other to form a handle housing around the medical probe handle.

The multifunctional enclosure as described herein may comprise an indication label that may be used to indicate if the medical probe contained therein is clean, sterile, dirty or used, for example. An indication label may be detachably attached to the multifunctional enclosure and may comprise a first label that is attached to the multifunctional enclosure, and a second label that is detachably attached to the first label. For example a second label may have the term “dean”, “sterile”, or “ready” to indicate that the medical probe enclosed therein is ready to be used for a procedure, and a first label, that is exposed after removal of the second label, may have the term “dirty”, “soiled, or “used” printed there on to indicate that the probe enclosed therein has been used or is contaminated. The second label may comprise a peel tab whereby it can be easily removed from the multifunctional enclosure. Medical personnel may be responsible for removing a second label after a procedure is complete and a contaminated probe is inserted back into the multifunctional enclosure. In addition, medical personnel may remove the first label prior to cleaning or sterilization of the multifunctional enclosure and may apply a new label or labels when the enclosure is cleaned and ready for use. A label may comprise any suitable term printed thereon, or may comprise an input field, whereby a message or code can be printed directly thereon. In addition, a label may comprise any other suitable type of tracking or identifying information. For example, a bar code may be configured on a label and this bar code may contain information about the probe inserted therein, the type of procedure or next location for transport of the multifunctional enclosure, and the like. A portion of a label may comprise ink or dyes that become visible or change color after being exposed to sterilization processing conditions including, heat, humidity, exposure to gases and the like, for example.

A multifunctional enclosure may be provided in a kit comprising a transport cart that is configured to retain at least one multifunctional enclosure. A transport cart as described herein, comprises at least one enclosure retainer feature for detachably attaching a multifunctional enclosure. An enclosure retainer feature may be a clip that is configured to receive the elongated sleeve portion of the multifunctional enclosure. A clip may expand to open and receive the elongated sleeve and hold the sleeve in position during transport. A transport cart may comprise two or more retainer features, such as one nearer the fist end and one nearer the second of the elongated sleeve. The transport car as described herein is configure with wheels whereby the cart can be moved from a procedure room to a cleaning facility, for example, without requiring the handling of the multifunctional medial probes.

The multifunctional enclosure as described herein may be used in a variety of ways. In one method of use, a medical probe or the elongated member of a medical probe may be inserted into the multifunctional enclosure as described herein, and the medical probe may be securely transported to and/or from, a medical procedural room, storage area, cleaning facility, or a sterilization procedure. In another embodiment, the multifunctional enclosure may be used as a reservoir for fluids that may be coated onto the medical probe prior or after a procedure. For example, the medical probe may be coated with a lubricating gel or warming fluid prior to a procedure by introducing the fluid into the multifunctional enclosure. The medical probe may then be removed from the multifunctional enclosure and inserted into a patient. In addition, the medical probe may be coated or treated with a cleaning solution or sterilization fluid after a procedure. For example a cleaning fluid, such as enzyme soap, for example, may be introduced into the multifunctional enclosure, and the medical probe may be inserted into the multifunctional enclosure for post procedural cleaning. Quickly treating the medical probe with a cleaning or sterilization fluid after a procedure may reduce the risk of cross contamination occurring and may provide for more effective and complete removal of biological material. Not immediately sterilizing a probe after a procedure is known as delayed reprocessing.

A multifunctional enclosure may be configured with a bendable segment along the length of the elongated sleeve, thereby allowing the elongated sleeve to be bent along the bendable segment without damage to the sleeve. In an exemplary embodiment, a bendable segment is configured between a first rigid segment and a second rigid segment. In an alternative embodiment, a bendable segment extends from an enlarged end of the elongated sleeve and a rigid segment is configured closer to the extended end, or further away from the medical probe insertion end. In still another embodiment, a bendable segment is configured after a rigid segment that extends from the medical probe insertion end, and extends substantially to the extended end. A rigid segment may comprise a material that is substantially rigid and that could not be bent 90 degrees without damage, such as kinking. A plastic tubing material may be a rigid segment, for example. A bendable segment may be any suitable type of material and/or configuration of material that allows bending without damage. For example, a bendable segment may comprise an elastomer or other pliable materials. In an exemplary embodiment, a bendable segment comprises a corrugated configuration and may be a corrugated section of the rest of the elongated sleeve. A corrugated segment or elastomeric segment may be attached to one or more rigid segments to form a multifunctional enclosure that may be bent and straighten thereafter without damage and/or kinking. A bendable segment along the elongated sleeve may provide for as much as about 90 degrees of bending or more, about 120 degrees of bending or more, about 180 degrees for bending or more, and any range between and including the bending amounts provided.

A bent elongated sleeve may be retained in a bent configuration by a bent retainer having a first capture portion for one portion of the elongated sleeve and a second capture portion for a second portion of the elongated sleeve. An extension between the first and second capture portions is configured to retain the elongated sleeve in a bent configuration. A bent retainer may be made out of a rigid or semi-rigid material, such as a plastic and one or more of the capture portions may have an opening to allow insertion of the elongated sleeve. One or more of the capture portions may be a ring that is configured to be slide over the elongated sleeve and extend completely around the elongated sleeve.

The summary of the invention is provided as a general introduction to some of the embodiments of the invention, and is not intended to be limiting. Additional example embodiments including variations and alternative configurations of the invention are provided herein.

### BRIEF DESCRIPTION OF THE DRAWINGS

The accompanying drawings are included to provide a further understanding of the invention and are incorporated in and constitute a part of this specification, illustrate embodiments of the invention, and together with the description serve to explain the principles of the invention.

### DETAILED DESCRIPTION OF THE ILLUSTRATED EMBODIMENTS

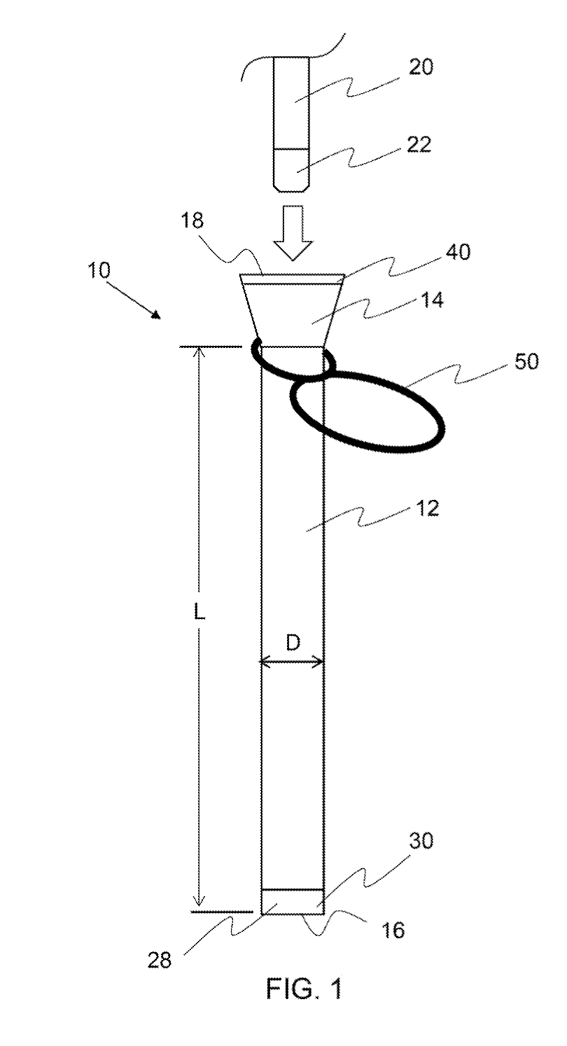

FIG. 1 shows a side view of a multifunctional enclosure described herein.

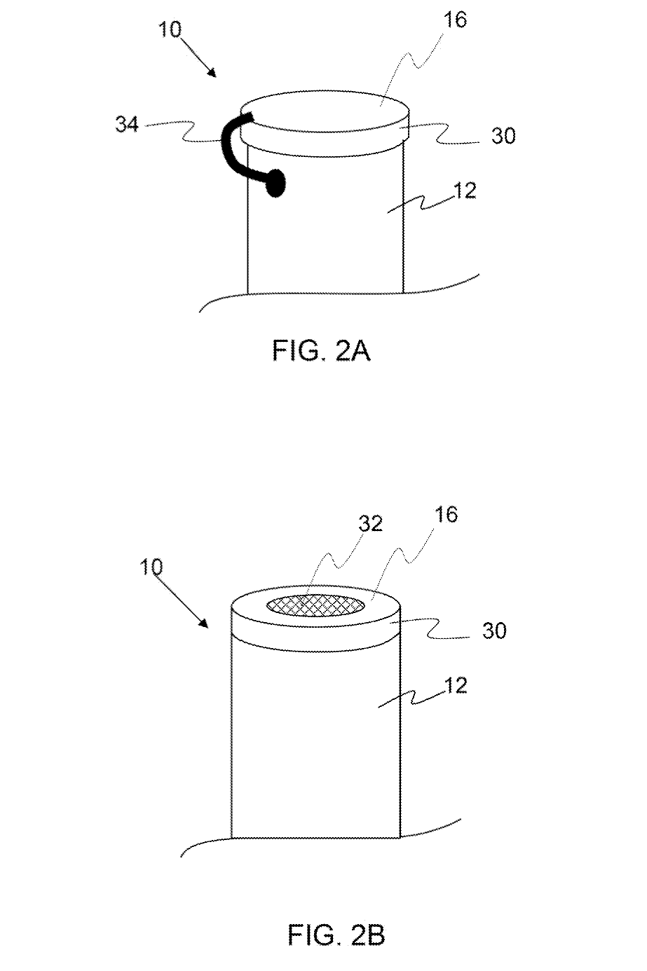

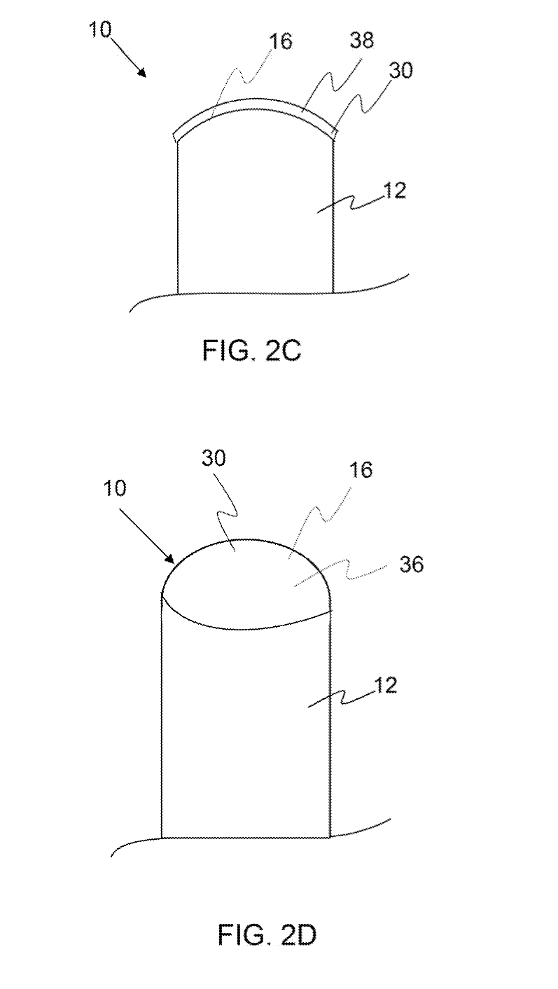

FIG. 2A shows an isometric view of the first end of a multifunctional enclosure described herein.

FIG. 2B shows an isometric view of the first end of a multifunctional enclosure described herein having a vent.

FIG. 2C shows a side view of the first end of a multifunctional enclosure described herein having a seam.

FIG. 2D shows an isometric view of the first end of a multifunctional enclosure described herein having molded end.

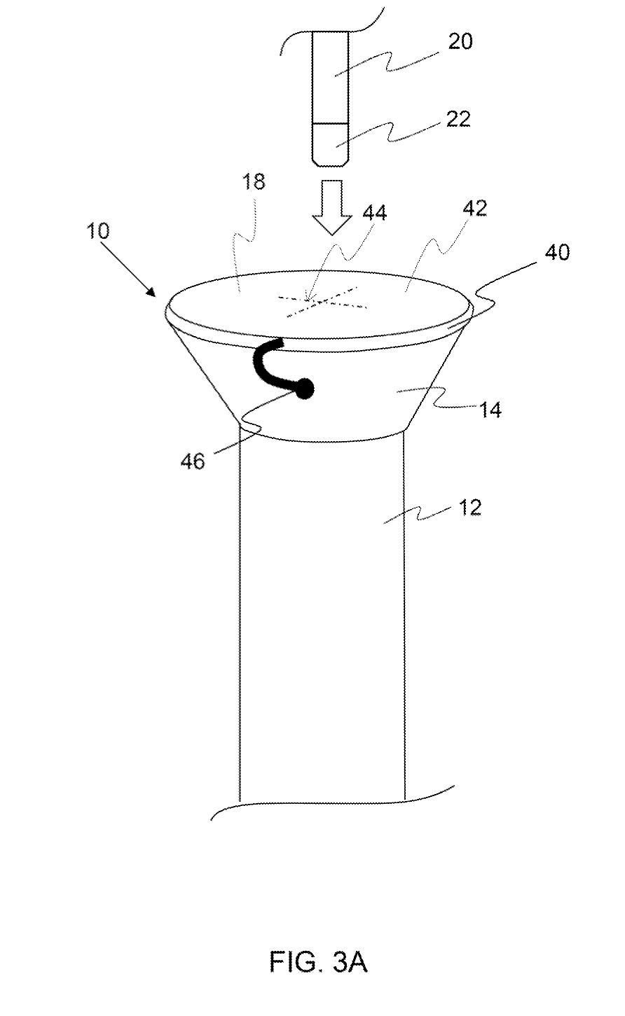

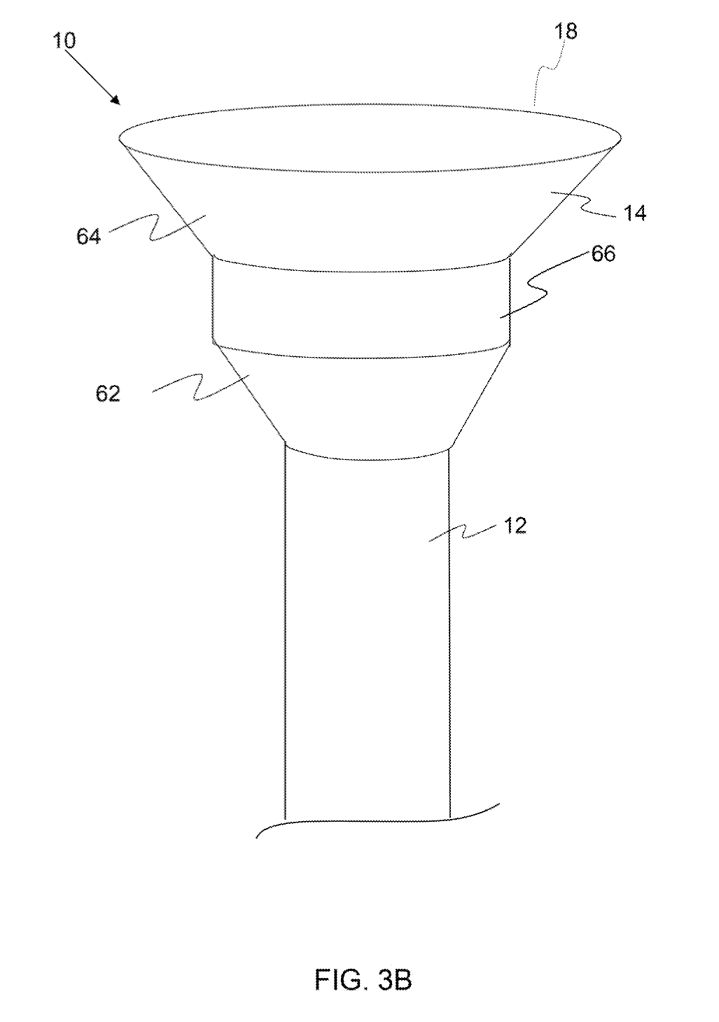

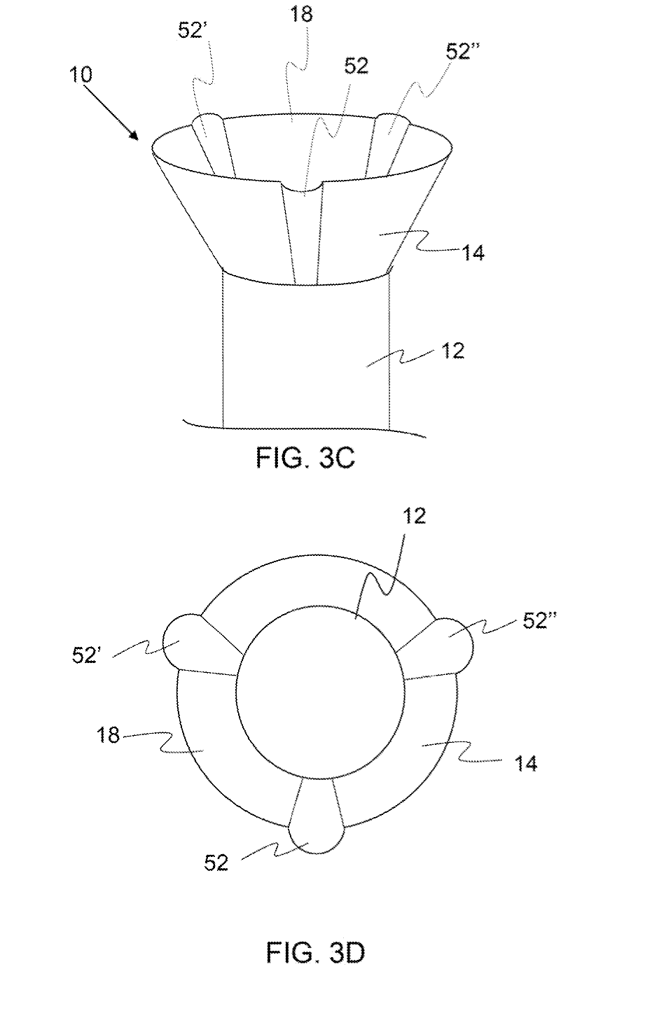

FIG. 3A shows an isometric view of the second end of a multifunctional enclosure described herein having a second end cover.

FIG. 3B shows an isometric view of the second end of a multifunctional enclosure described herein.

FIG. 3C shows an isometric view of the second end of a multifunctional enclosure described herein having flutes.

FIG. 3D shows a top down view of the second end of a multifunctional enclosure described herein having flutes.

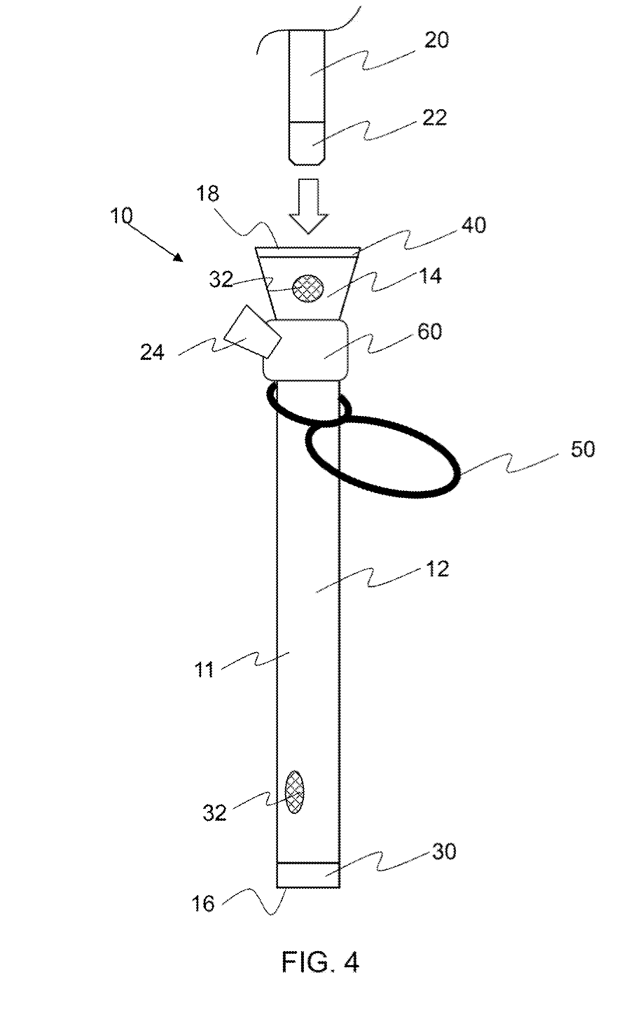

FIG. 4 shows a side view of a multifunctional enclosure described herein having an attachment component and two vents.

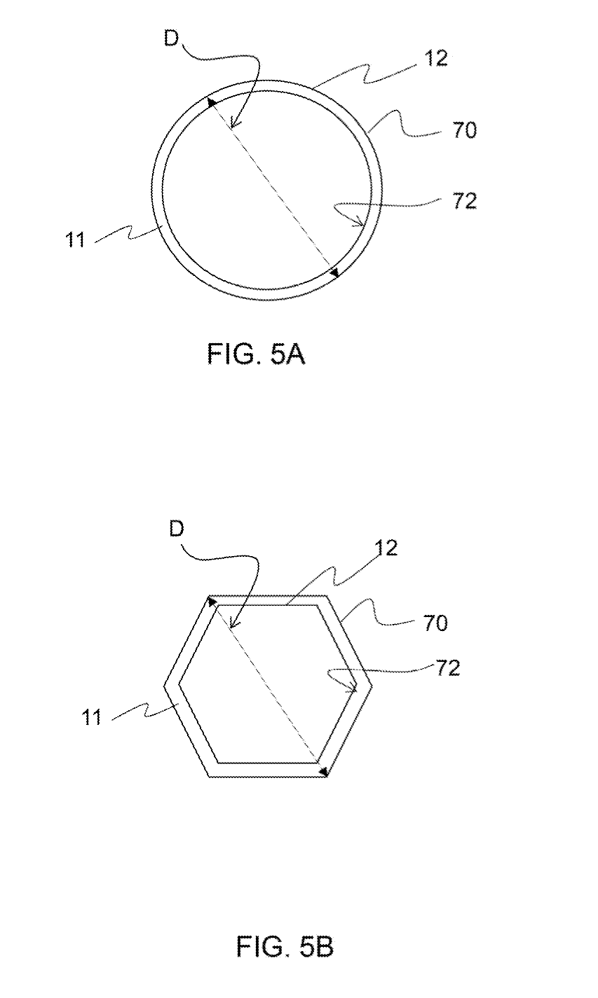

FIG. 5A show a cross sectional view of the elongated sleeve described herein.

FIG. 5B show a cross sectional views of the elongated sleeve described herein.

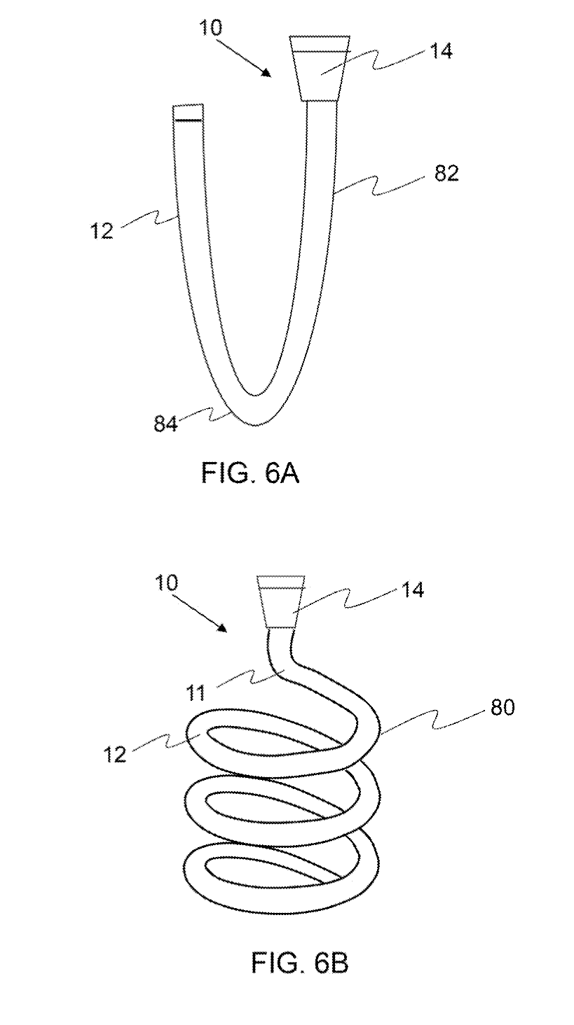

FIG. 6A shows a side view of a multifunctional enclosure described herein having a “U” shaped elongated sleeve.

FIG. 6B shows a side view of a multifunctional enclosure described herein having a helically shaped elongated sleeve.

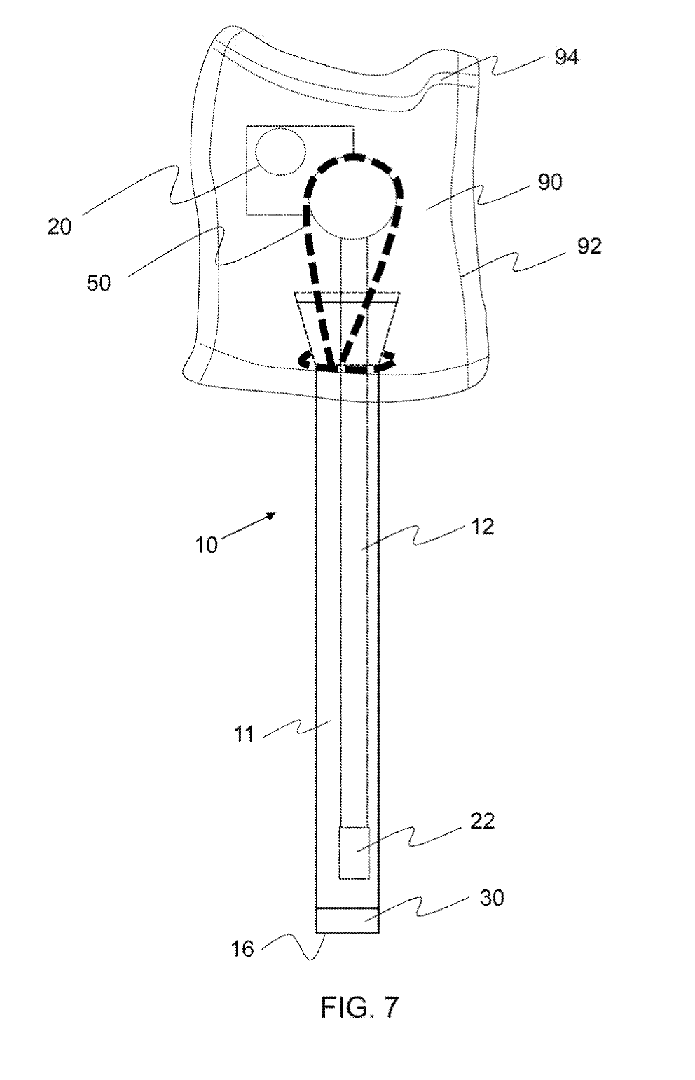

FIG. 7 shows a side view of a multifunctional enclosure described herein having an enclosure pouch.

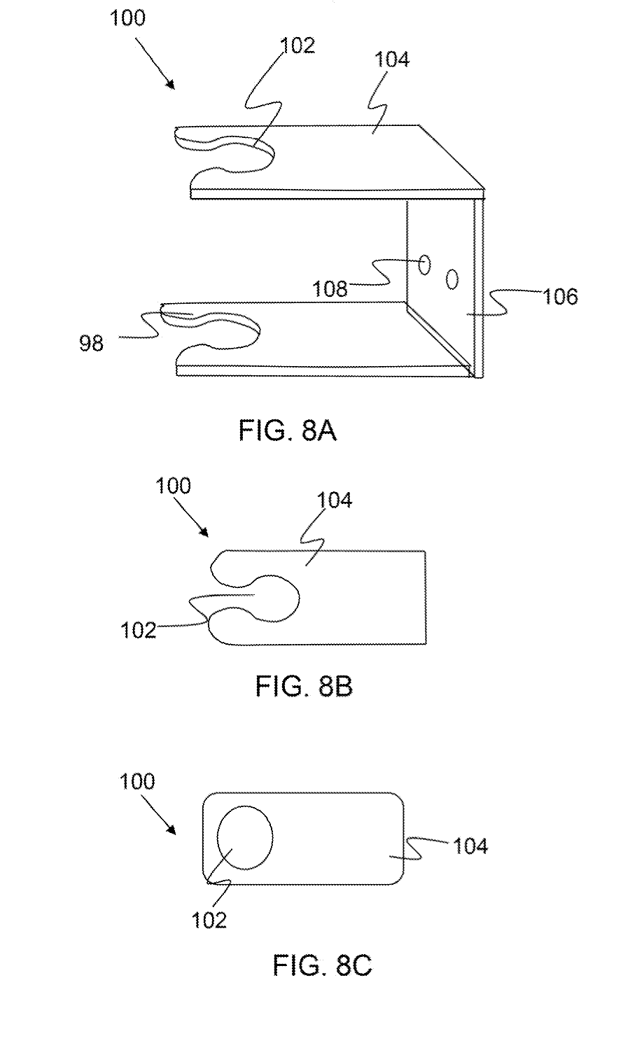

FIG. 8A shows an isometric view of a bracket described herein.

FIGS. 88 and 8C show top views of brackets described herein.

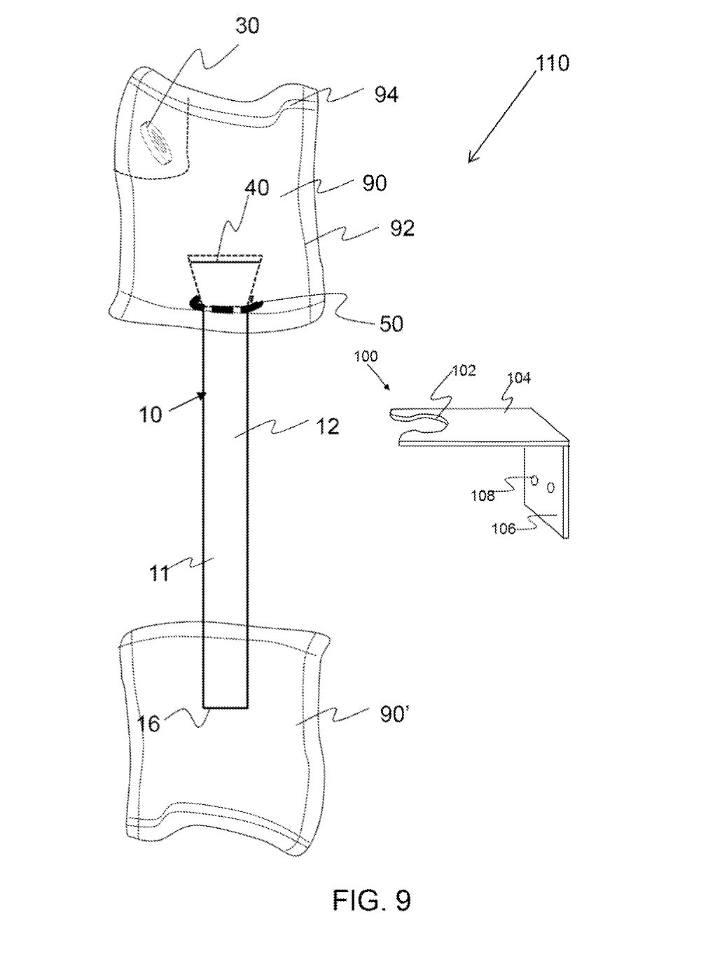

FIG. 9 shows an isometric view of a multifunctional enclosure kit described herein.

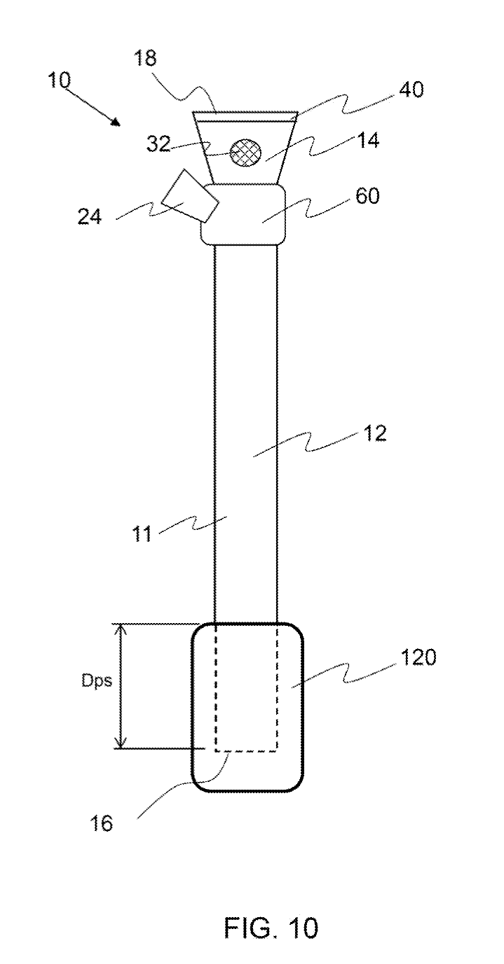

FIG. 10 shows a side view of an exemplary multifunctional enclosure having a protective feature.

FIG. 11 shows a side view of an exemplary multifunctional enclosure having a protective feature.

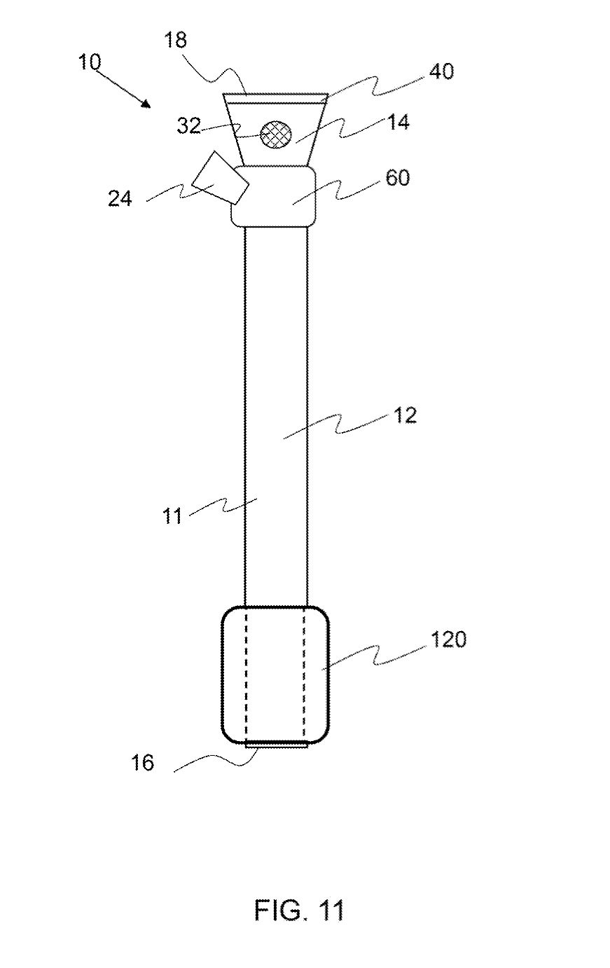

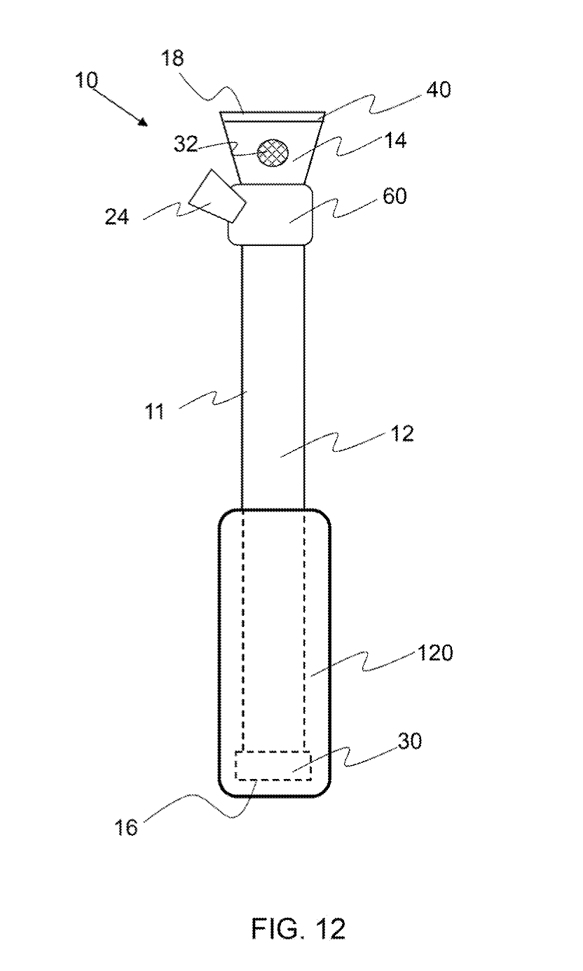

FIG. 12 shows a side view of an exemplary multifunctional enclosure having a protective feature.

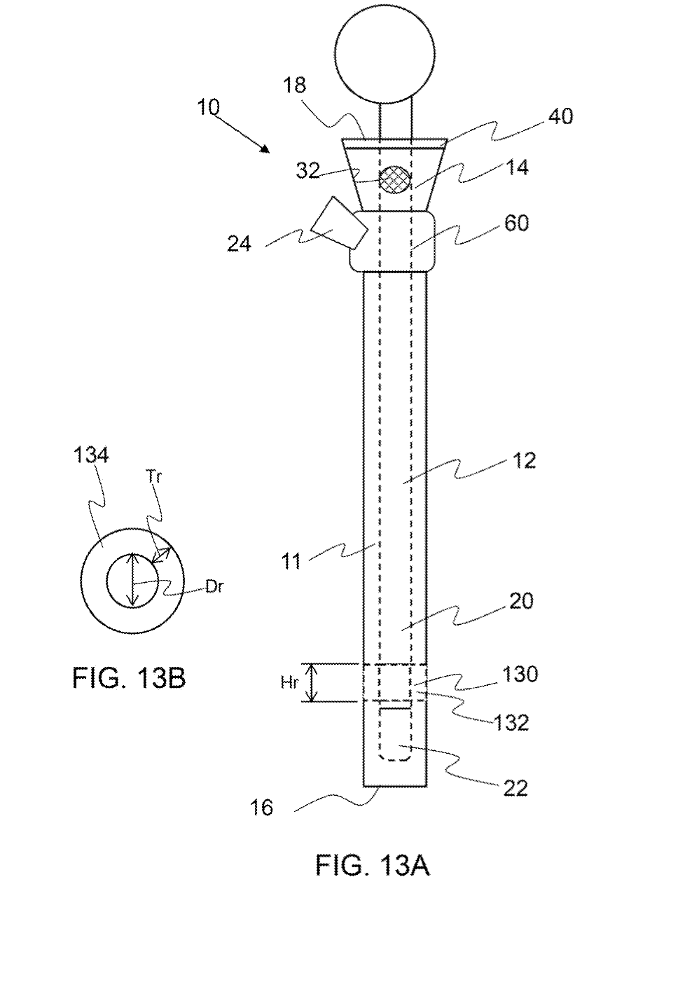

FIG. 13A shows a side view of an exemplary multifunctional enclosure having a probe tip retainer.

FIG. 13B shows a top-down view of an the probe tip retainer shown in FIG. 13A.

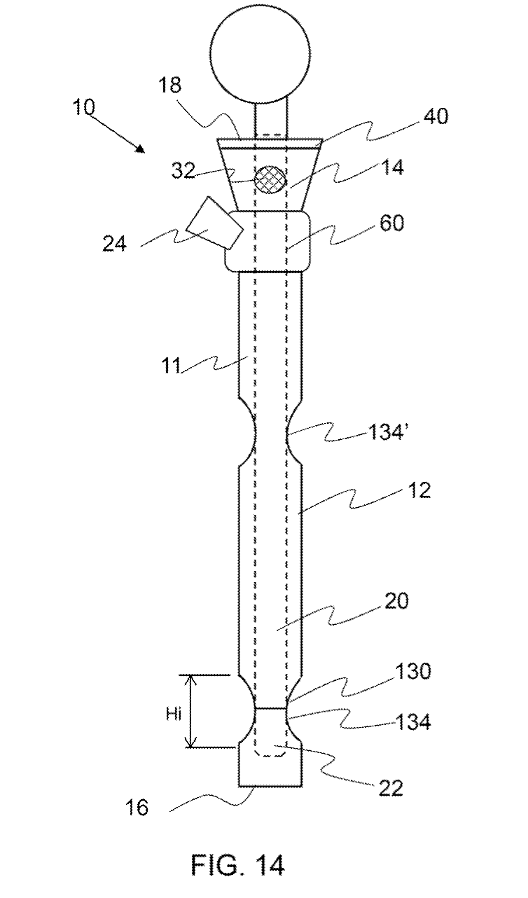

FIG. 14 shows a side view of an exemplary multifunctional enclosure having a probe tip retainer.

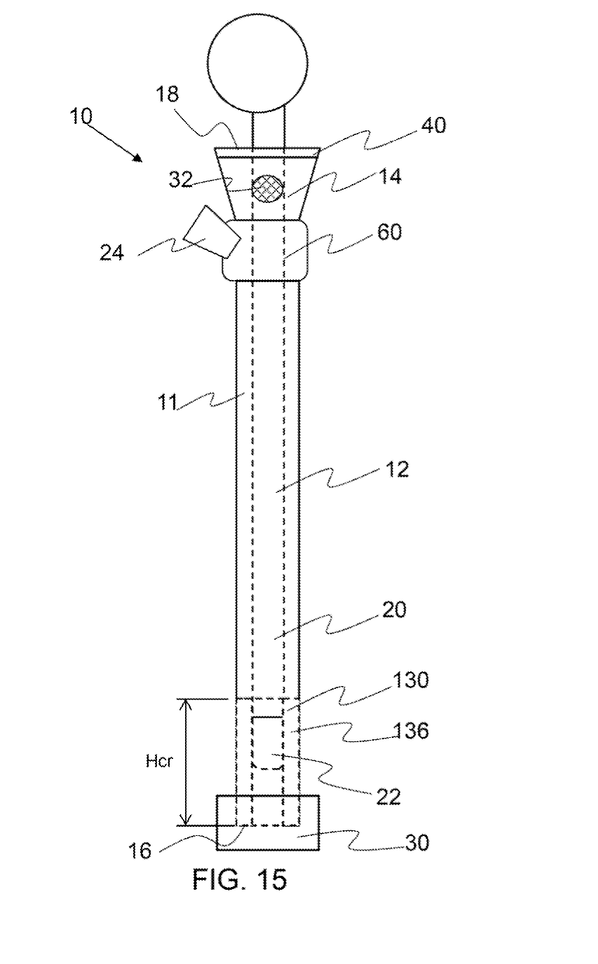

FIG. 15 shows a side view of an exemplary multifunctional enclosure having a probe tip retainer.

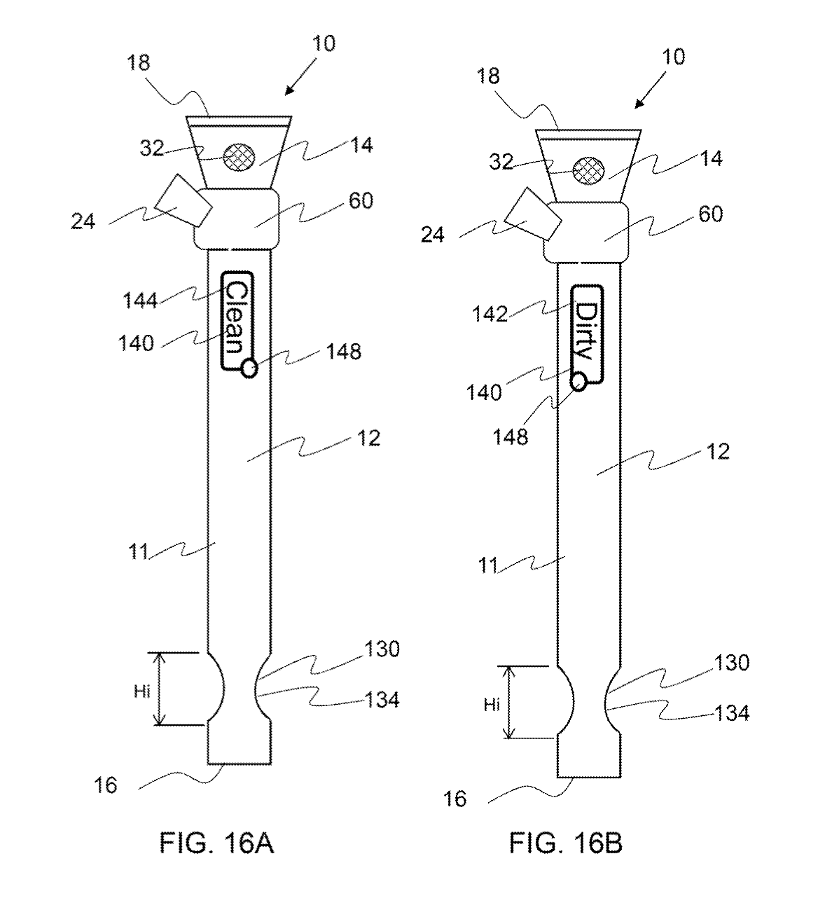

FIG. 16A shows a side view of an exemplary multifunctional enclosure having a label attached to the elongated sleeve.

FIG. 16B shows a side view of an exemplary multifunctional enclosure having a label attached to the elongated sleeve.

FIG. 17 shows an isometric view of an exemplary multifunctional enclosure transport kit comprising a transport cart.

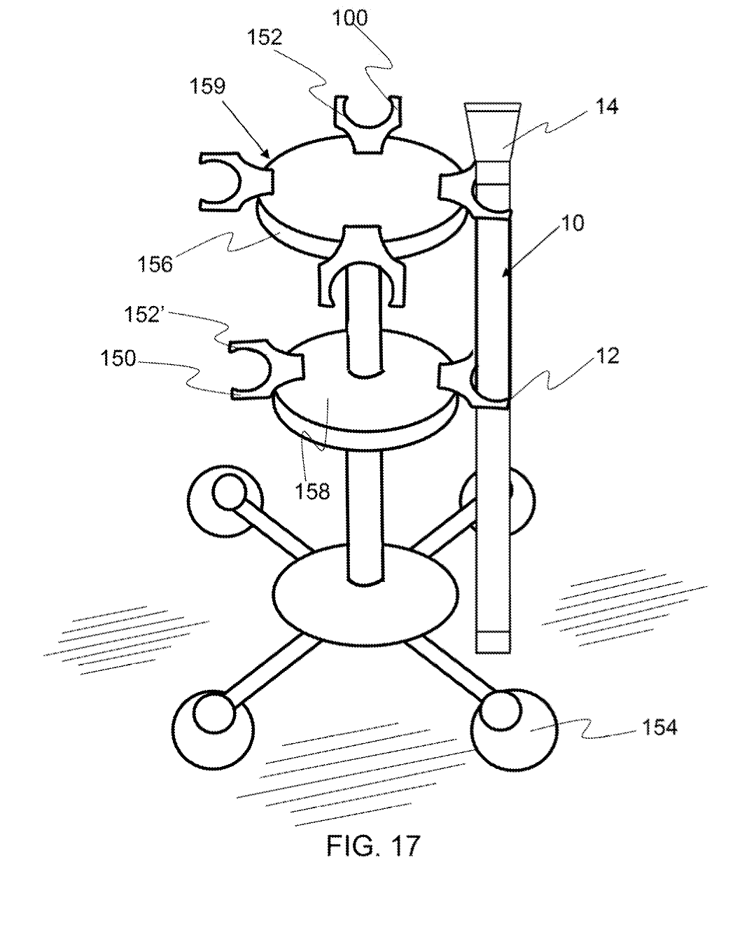

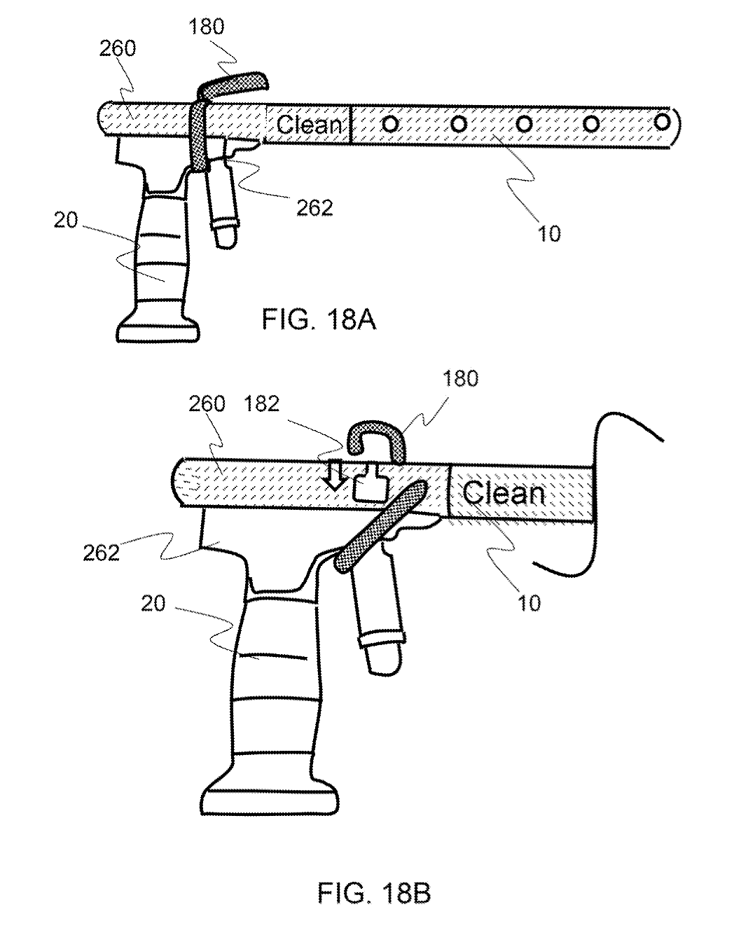

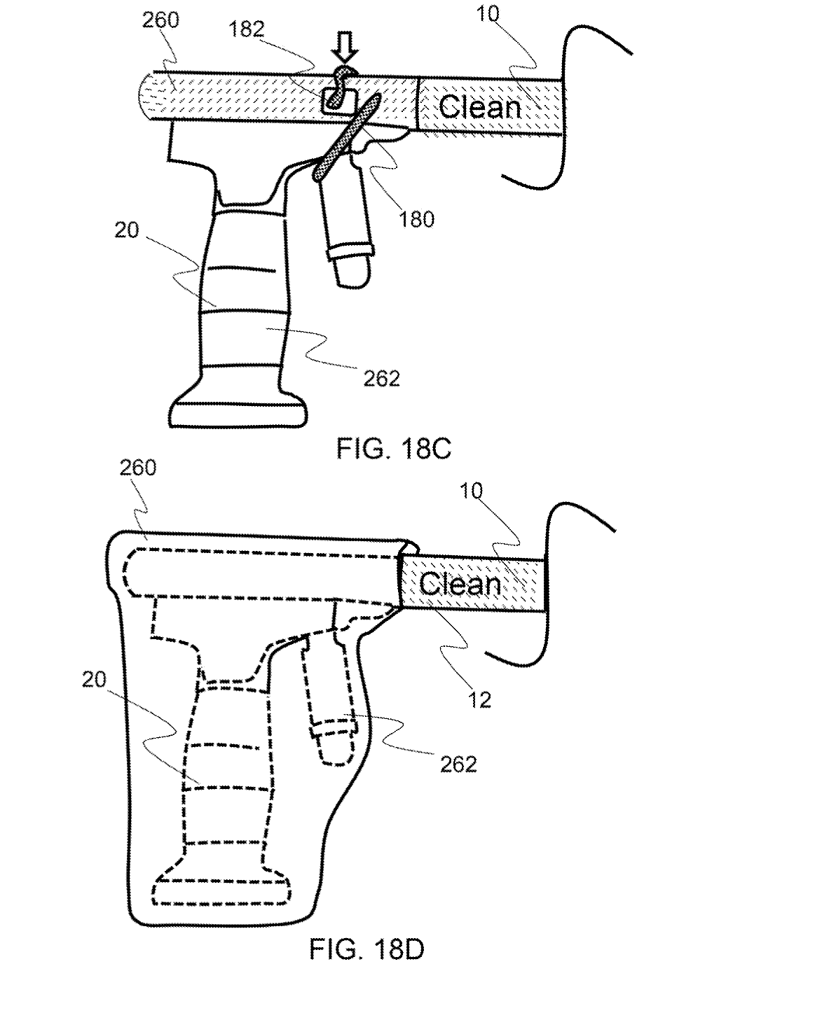

FIGS. 18A-18D show side views of a medical probe configured for neurosurgery and attached to a multifunctional enclosure as described herein.

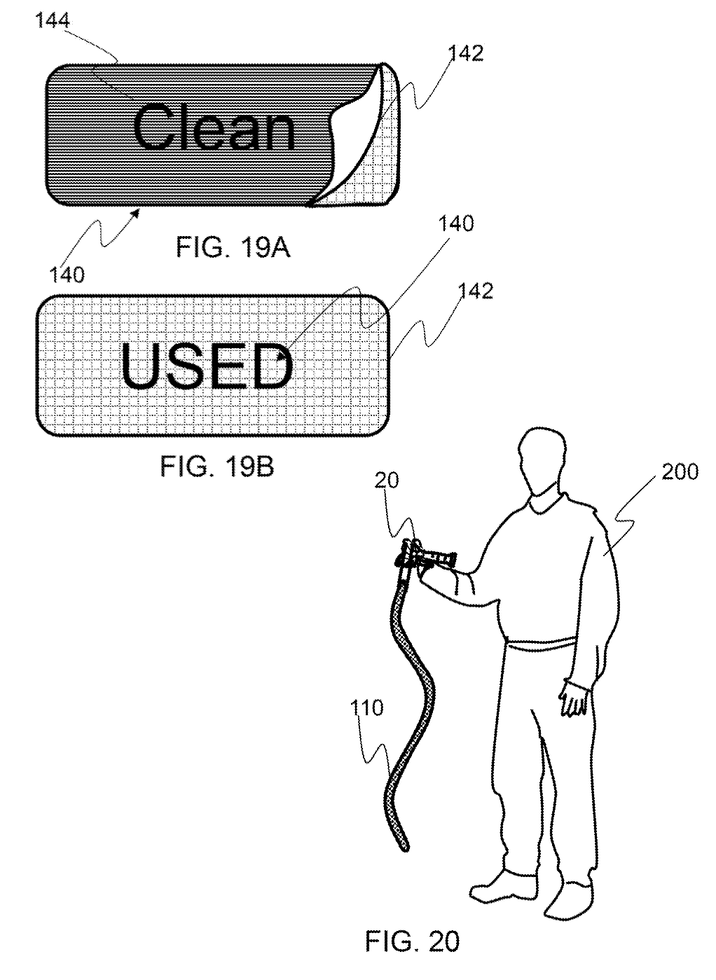

FIGS. 19A and 19B shown top-down views of an exemplary label comprising a first and second label detachably attached.

FIG. 20 shows a person holding a medical probe having a long and flexible elongated probe member as described herein.

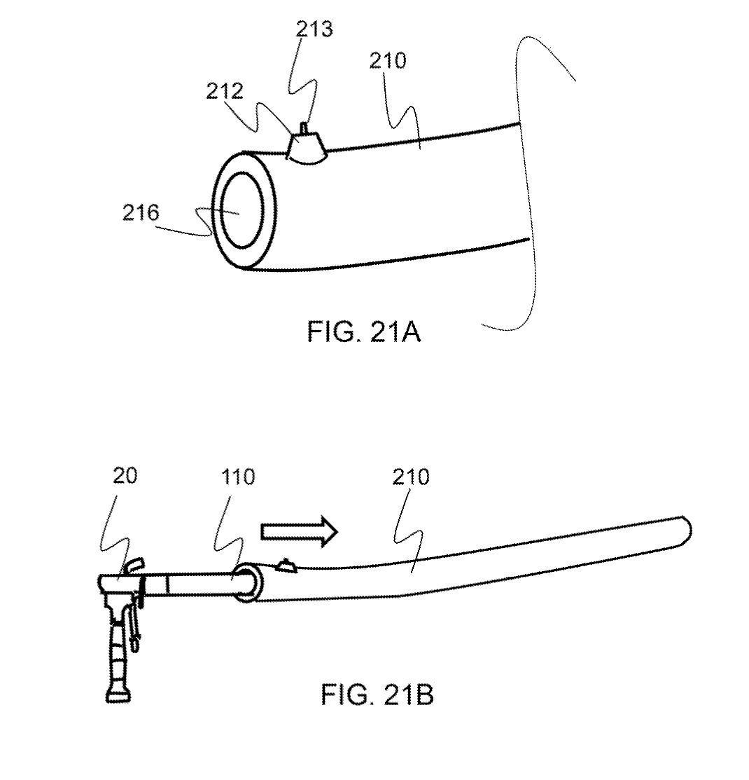

FIG. 21A shows an isometric view of an exemplary inflatable support sleeve having a release valve.

FIG. 21B shows an isometric view of the elongated probe member of a medical probe being inserted into an exemplary inflatable support sleeve.

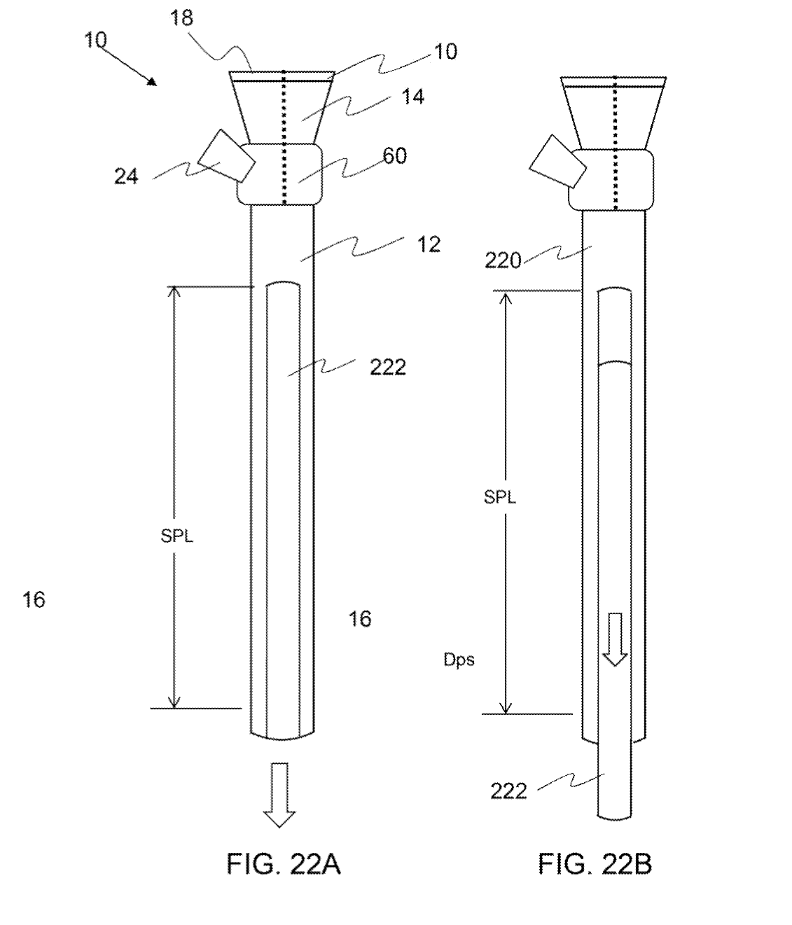

FIGS. 22A and 22B show side views of an exemplary multifunctional enclosure having a detachable sleeve portion.

FIGS. 23A and 23B show side views of an exemplary multifunctional enclosure having a detachable elongated sleeve having a detachable sleeve portion.

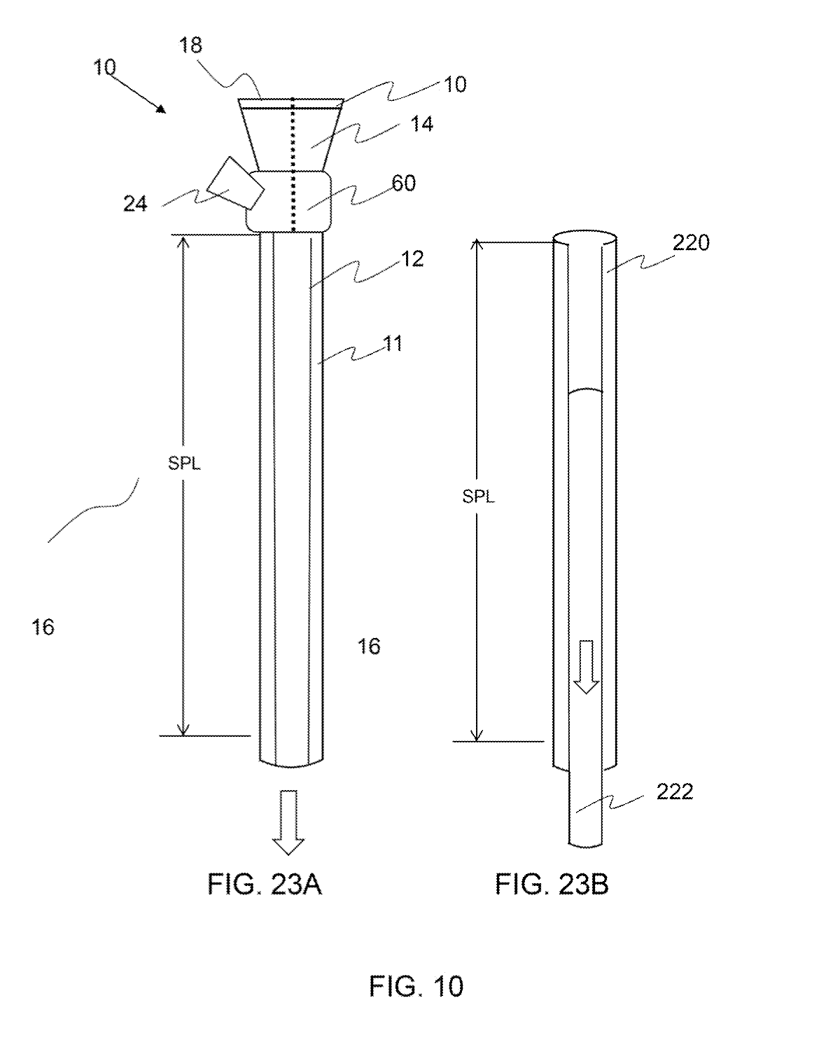

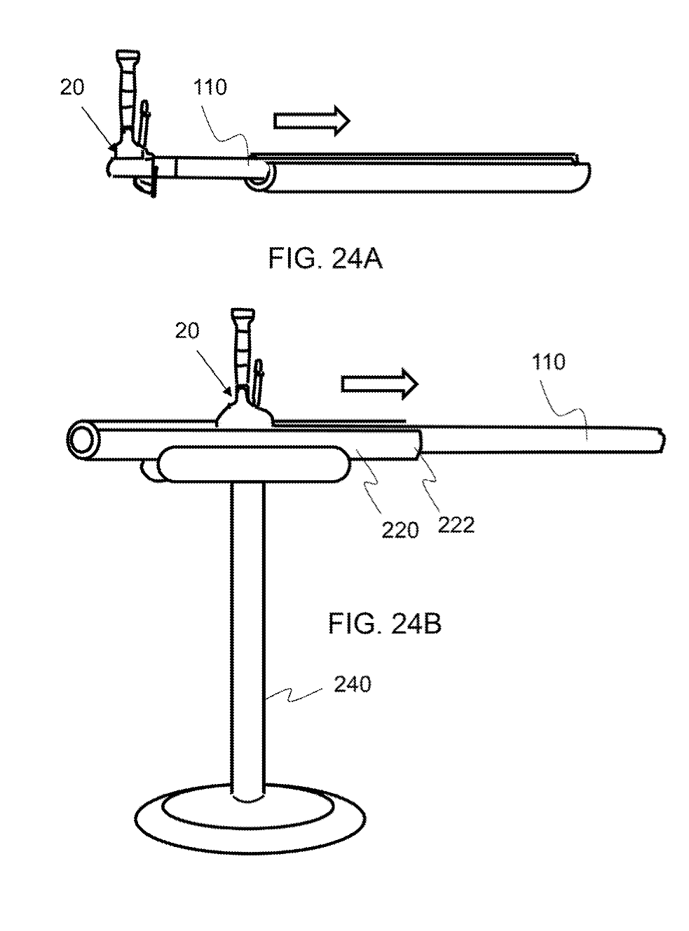

FIGS. 24A and 24B show isometric side views of an elongated probe member of a medical probe being supported by an exemplary detachable elongated sleeve having a detachable sleeve portion.

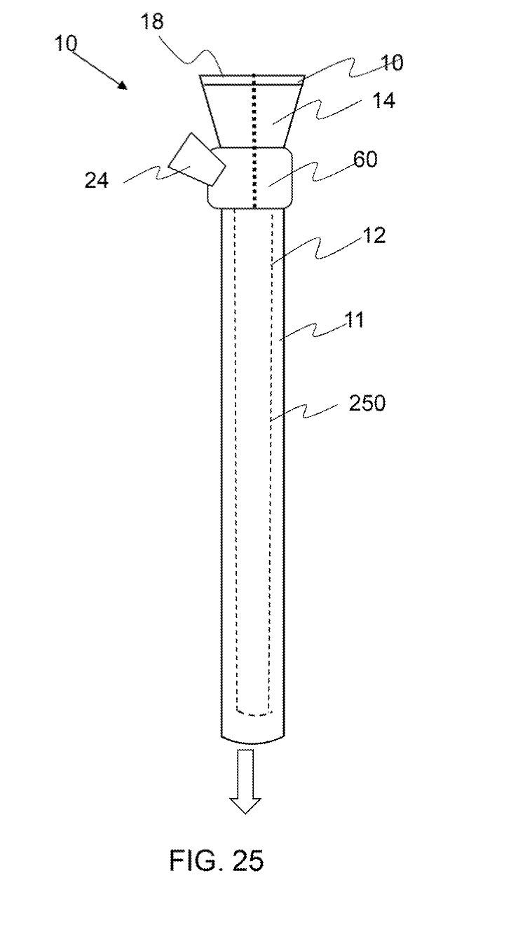

FIG. 25 shows a side view of an exemplary multifunctional enclosure having a sleeve liner configured therein.

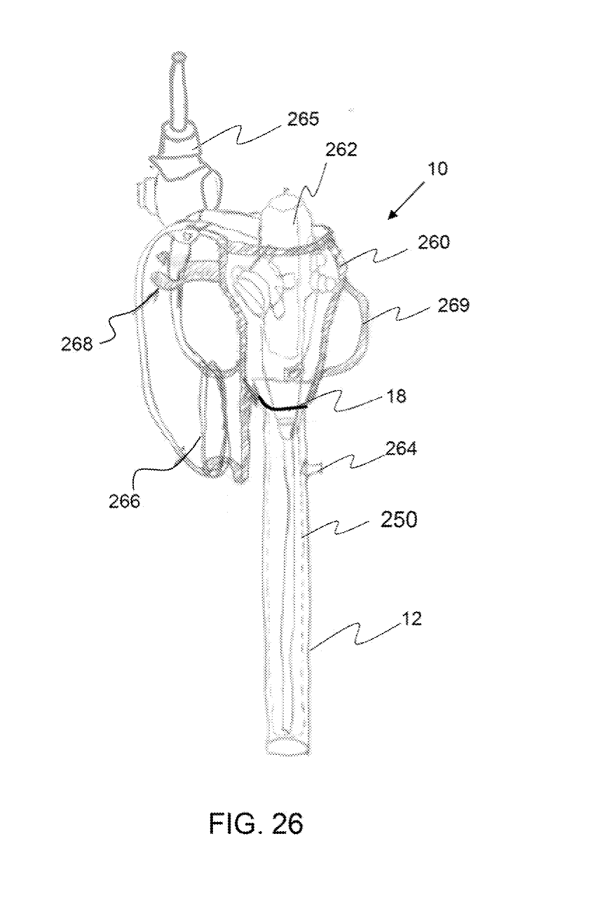

FIG. 26 shows an isometric view of an exemplary multifunctional enclosure comprising a handle housing and an elongated sleeve liner.

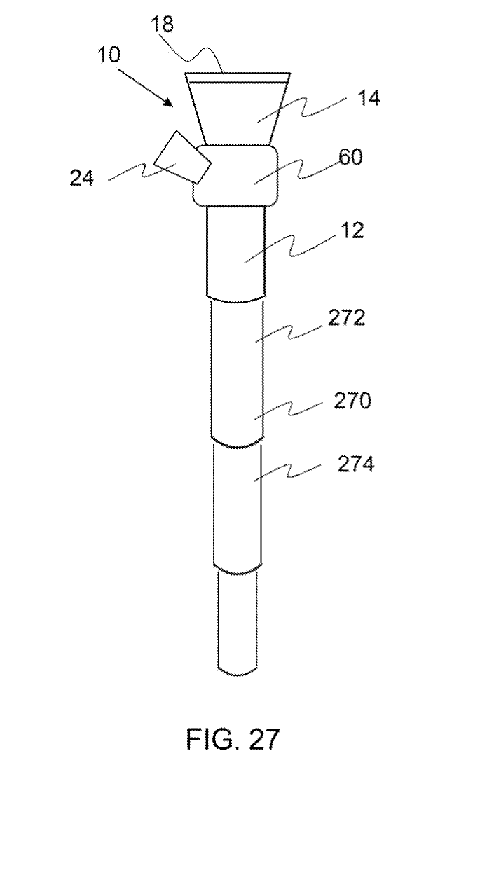

FIG. 27 shows an isometric view of an exemplary telescoping multifunctional enclosure.

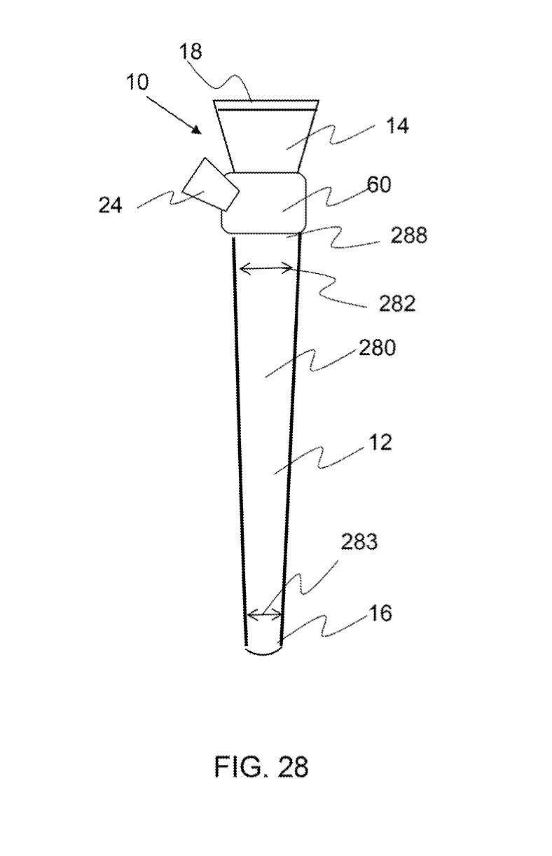

FIG. 28 shows a side view of an exemplary funnel shaped multifunctional enclosure.

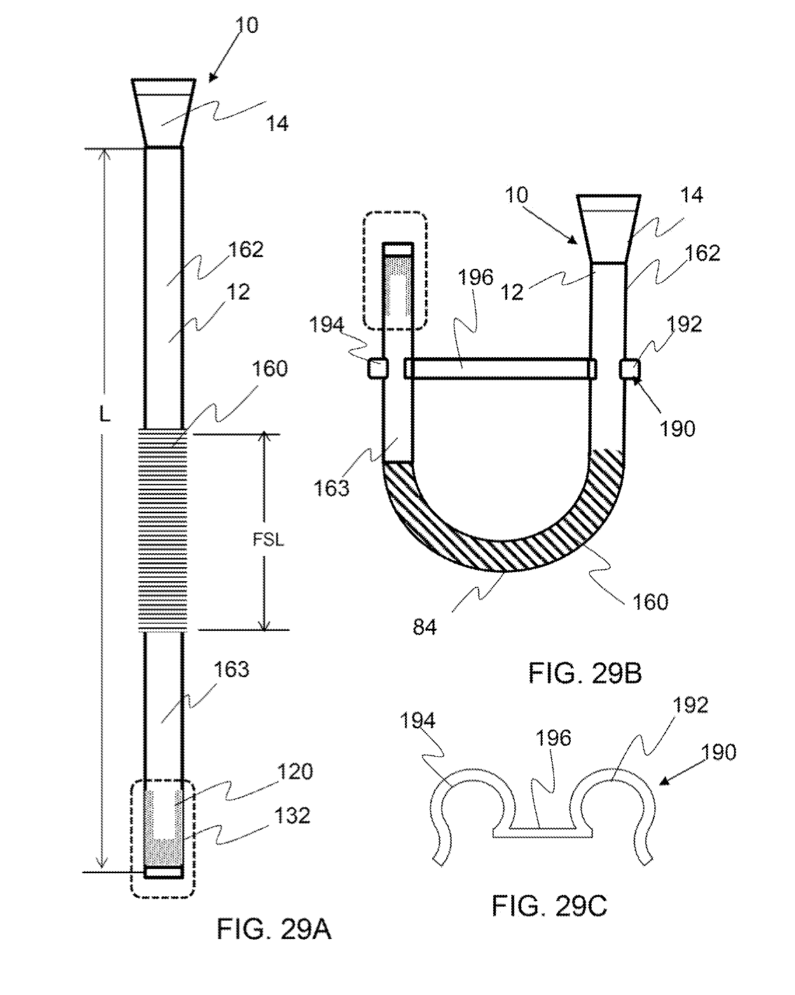

FIG. 29A shows a side view of an exemplary multifunctional enclosure having a bendable segment configured between a first rigid segment and a second rigid segment, in a straight configuration.

FIG. 29B shows a side view of an exemplary multifunctional enclosure having a bendable segment configured between a first rigid segment and a second rigid segment, in a bent configuration.

FIG. 29C shows a top-down view of an exemplary bent retainer that is configured to retain an elongated sleeve in a bent configuration.

Reference numbers to elements are used consistently throughout the figures; however, the elements may have different embodiments such as shape, or configuration, for example.

The invention is directed to a multifunctional enclosure for medical probes and in particular the elongated members of a medical probe. As shown in FIG. 1, the multifunctional enclosure 10 comprises an elongated sleeve 12 having a first end 16, a second end 18 and an attachment component 50. In this embodiment, the first end comprises a first end closure portion 30, and the elongated member of the medical probe may be inserted through the second end. The elongated sleeve 12 may be rigid as defined herein, in order to prevent the elongated member of the medical probe from swinging or moving during transport or handling. The elongated sleeve may be made out of any suitable material including but not limited to plastic, metal, glass, composites, combinations of said materials, and the like. The elongated sleeve may be transparent or translucent, opaque or a specific color to indicate a particular type of multifunctional enclosure. In some embodiments, the multifunctional enclosure, or the elongated sleeve of the multifunctional enclosure may be transparent to allow for easy recognition of the medical probe inside. In addition, a transparent or translucent multifunctional enclosure, may allow a user to determine the level of cleaning or other fluid introduced into the multifunctional enclosure.

In one embodiment, the elongated sleeve is a tube, such as a plastic tube. The elongated sleeve may be configured in a generally straight shape, or comprise at least one bend or curve. In addition, the elongated sleeve as described herein may be a “U” shape 82 as shown in FIG. 6A having a bend 84. In another embodiment, the elongated sleeve, or a portion of the elongated sleeve 12 may be configured into helical shape. The helical elongated sleeve 80, may comprise one or more generally straight sections as shown in FIG. 6B.

The elongated sleeve may have an aspect ratio of length L over maximum outer dimension, or outer diameter D, as shown in FIG. 1, greater than about 3, or more. The maximum outer dimension D is the maximum dimension that can be measured across the cross section of the elongated member as shown for example in FIGS. 5A and 5B. When the elongated member is a tube, the maximum outer dimension is simply the outer diameter of the tube as shown if FIG. 5A. When the elongated member is a polygonal or irregular shape it is the maximum dimension measured as shown in FIG. 5B. The aspect ratio of the elongated sleeve may be any suitable value such as but not limited to greater than about five, greater than about eight, or greater than about ten, greater than about twenty, greater than about fifty, to accommodate various types of medical probe.

The length of the elongated member of the multifunctional enclosure can be any suitable length to accommodate the medical probe and may be for example greater than about 6 in, greater than about 12 in, greater than about 20 in, greater than about 40 in, greater than about 60 in greater than about 80 in, or any range between lengths described including for example from about 6 in to 80 in, or 6 in to 60 in.

The diameter or maximum outer dimension of the elongated member of the multifunctional enclosure may be any suitable dimension to accommodate the medical probe and may be for example greater than about 0.1875 in, greater than about 0.25 in, greater than about 0.5 in, greater than about 1.0 in, greater than about 1.25 in, greater than about 1.5 in greater than about 2.0 in, or any range between lengths described including for example from about 0.1875 in to 2.0 in, or 0.25 in to 1.5 in.

The elongated sleeve of the multifunctional enclosure, may have any suitable cross sectional shape, such as a circular shape as shown in FIG. 5A, or a polygon shape, as depicted in FIG. 5B as a hexagonal shape. Any suitable shape may be used, and in some embodiments, an irregular or polygonal shape may be used to avoid creating a seal against the medical probe at the second end. The elongated sleeve 12 is configured such that the medical probe 20, may easily slide in and out as shown in FIG. 1. The medical probe may be positioned within the elongated sleeve with a sufficient clearance from the inside surface 72.

The first end 16 of the multifunctional enclosure 10 may comprise a first end closure portion 30, such as a detachable cap 28, as shown in FIG. 1. The first end closure portion may be removed during cleaning or sterilization procedures to allow for flow through the elongated sleeve. In addition, the first end closure portion may be removed to allow for adequate drying after cleaning or sterilization. A first end closure attachment portion 34, as shown in FIG. 2A, may allow the first end cover to be removed from the end without being completely removed from the multifunctional enclosure. The first end closure attachment portion may comprise any suitable means to attach the first end closure portion 30 to the multifunctional enclosure 10. As shown in FIG. 2A, the first end closure attachment portion 34 comprises a piece of material attached to the first end cover at one end and attached to the elongated member at the opposing end.

The first end closure portion may be a cap that extends over the outer surface of the elongated member, or may be a plug that comprises a portion that is inserted into the opening of the first end. The first end closure portion may be configured to be detachably attached to the fist end and may comprise threads such that the first end portion may be screwed onto the first end. Threads may be configured onto the inside and/or outside surface of either the first end closure portion or the first end. In another embodiment, the first end closure portion may be pushed onto the first end and may be designed with tolerances to adequately seal the first end. In addition, the first end closure portion may be snapped onto the first end, and either the closure portion or the first end may comprise at least one raised element, or ridge that provides an interference fit for the closure portion to be snapped on. In yet another embodiment, the first end closure portion may expand or contract to seal the first end. The first end closure portion may comprise an elastomer to secure the closure portion to the first end.

In yet another embodiment the first end cover portion comprises a seam 38 where the first end 16 of the multifunctional enclosure 10 has been sealed as shown in FIG. 2C. For example, the first end of a plastic multifunctional enclosure may be heat pressed and welded together for form a seal and first end clover portion. In still another embodiment, the first end 16 of the multifunctional enclosure 10 may comprise a molded end 36, as shown in FIG. 2D. The molded end may be configured in any suitable shape, such as a rounded or spherical shape.

In one embodiment, the first end, or first end cover 30 comprises a vent 32 as shown in FIG. 28. A vent however, may be configured anywhere on the multifunctional enclosure 10 as shown in FIG. 4. The vent may be antimicrobial, and/or hydrophobic. In one embodiment, the vent comprises a porous material that meets standards for antibacterial ratings, such as materials having a log reduction value (LRV) of more than 99.999 when tested according to ASTM F1608. The vent may have sufficient air flow to allow for adequate drying of the medical probe after cleaning, sterilization, or during storage. The vent may comprise a material that has a gurley value as measured with a Gurley Densometer 4380, of no more than 100 seconds, and may have gurley values of no more than about 5 seconds, no more than about 20 seconds, no more than about 50 seconds. The vent material may comprise expanded polymers such as expanded polytetrafluoroethylene membrane, microfiber materials, sintered polymers such as Zitex membranes available from Saint Gobain Inc., Worchester, Mass., SureVent membranes available from Millipore Inc., Billerica, Mass., and the like. The hydrophobic vent may allow fluid to be poured into the enclosure and retained while also allowing for adequate air flow during sterilization and/or drying.

The second end 18 or opening of the multifunctional enclosure 10 may comprise an enlarged end 14, or configured to facilitate the insertion of the medical probe, as shown in FIG. 3A. The second end may be any suitable shape such as a funnel or bell shape. As shown in FIG. 3A the enlarged end 14 is in a funnel shape, where the second end 18 is larger in diameter than the elongated sleeve 12. As shown in FIG. 3B, the enlarged end 14 comprises a plurality of shapes, including a first funnel shape portion 62 and a second funnel shape portion 64. A straight shape portion 66 connects the two funnel shape portions as shown in FIG. 3B.

The enlarged portion 14 may further comprise flutes 52, 52′ and 52″ as shown in FIGS. 3C and 3D. The flutes may be configured into the enlarged portion to allow the passage of air when a medical probe is inserted into the multifunctional enclosure. In some embodiments, the medical probe may fit snugly into the second end of the multifunctional enclosure and effectively prevent air passage through the second end into the elongated sleeve. The flutes may have any suitable shape such as a curved or angled shape. The flutes may extend the entire length of the enlarged portion, or only a portion of the length of the enlarged portion. In one embodiment, a flute or flutes may extend along a portion of the elongated sleeve.

In one embodiment, the second end comprises a cover 40 that may be removed to allow for the insertion of the medical probe. In an alternative embodiment, the second end cover 40 may comprise an opening 44, such as a hole or at least one slit, whereby the medical probe 20 may be inserted, as shown in FIG. 3A. The second end cover may protect the interior of the multifunctional enclosure from contamination prior to use. In one embodiment, the multifunctional enclosure comprises two second end covers; one being solid, and the other having an opening. In this embodiment, the solid second end cover may be removed and the medical probe may be inserted through the opening in the second end cover. The second end cover may also be attached to the multifunctional enclosure by a second end cover attachment portion 46, as shown in FIG. 3A.

The attachment component 50, as shown in FIG. 1 may comprise any number of components configured to attach the multifunctional enclosure 10 to the medical probe 20. The attachment component may include for example, elastic bands, hooks, latches, hook and loop fasteners and the like. In one embodiment, the attachment component comprises at least one elastic band that may be detachably attached to the medical probe, such as by stretching the band and locating the stretched end over a portion or projection of the medical probe.

The multifunctional enclosure 10 may further comprise an enclosure pouch 90 that may be configured to enclose the medical probe 20 as shown in FIG. 7. In one embodiment, the enclosure pouch 90 is attached to the multifunctional enclosure 10, and comprises a sealing portion 94 along the extended end that may be used to seal the medical probe 20 within the enclosure pouch as shown in FIG. 7. The medical probe 20 is further restrained by the attachment component 50, that is looped around a portion of the medical probe as shown in FIG. 7. The pouch may comprise any suitable material including but not limited to plastic, metal foil, fabric, antimicrobial materials, such as porous or microporous materials, combinations of material, and the like. The pouch 90 may be a sterilization pouch, and may comprise a porous side and a non-porous side. For example, the pouch may comprise a paper side and a plastic side. The sealing portion of the pouch 94, may comprise any suitable sealing mechanism or material, including but not limited to a zipper, hook and loop fastener, adhesive, interference fit seal, thermally welded seal, and the like.

In one embodiment, both the first and second end of the multifunctional enclosure comprise a pouch 90 and 90′ that may be removed prior to using the multifunctional enclosure as shown in FIG. 9. The pouches may be attached to one or both ends of the multifunctional enclosure to prevent contamination of the interior of the multifunctional enclosure. The pouches may be attached to the multifunctional enclosure through any suitable means including but not limited to tape, adhesives, heat welding, ultrasonic welding and the like.

The multifunctional enclosure describe herein may be positioned and temporarily restrained by a bracket 100, as shown in FIGS. 8A, 8B and 8C. The bracket 100 as shown in FIG. 8A, may have an opening 102 configured in a first member to accept the multifunctional enclosure. The multifunctional enclosure may slide direct into the bracket and the enlarged end of the multifunctional enclosure may support the enclosure. A second member 106 may be attached to the first member 104 of the bracket as shown in FIG. 8A. In addition, the bracket may further comprise at least one attachment portion, such as a hole or holes 108 as shown in FIG. 8A. The opening 102 in the bracket 100, may be configured in any suitable manner to accept and support the multifunctional enclosure as described herein. A bracket may configured with a second portion 98 that is similar to the first member 104, and positioned at some length down along the attachment portion, thereby providing two opening for retaining the elongated sleeve.

The multifunction enclosure as describe herein may be provided as a kit 110 as shown in FIG. 9 The kit 110 may include at least one multifunctional enclosure 10, and a bracket 100. The kit may further include at least one pouch 90 attached to at least one end of the multifunctional enclosure, an attachment component 50, a first end closure portion 30, and a second end cover 40. The first end closure portion 30 may be attached to the first end, or it may be within one of the pouches 90 as shown in FIG. 9.

As shown in FIG. 10, an exemplary multifunctional enclosure 10 has a protective feature 120 configured on the first end 16 of the elongated sleeve 12. The protective feature extends around the first end 16 and up along the elongated sleeve 12 a distance Dps as shown in FIG. 10. The distance Dps may be any suitable distance including, substantially the entire length of the elongated sleeve, less than 50% of the length of the elongated sleeve, less than 25% of the length of the elongated sleeve, less than 10% of the length of the elongate sleeve and any range between and including the length values provided. The protective feature may be a comprised of a soft recoverable material such as foam, or elastomer. In an exemplary embodiment the protective feature is a soft urethane foam that can be detachably attached to the elongated sleeve.

As shown in FIG. 11, an exemplary multifunctional enclosure 10 has a protective feature 120 configured around the elongated sleeve 12 at the first end 16.

As shown in FIG. 12, an exemplary multifunctional enclosure 10 has a protective feature 120 that is attached to and part of the first end closure portion 30. This type of protective feature would be detached when the first end closure portion was removed from the first end of the elongated sleeve.

As shown in FIG. 13A, an exemplary multifunctional enclosure 10 has a probe tip retainer 130 configured with the elongated sleeve 12. The probe tip retainer is a retainer insert type 132 whereby it reduces the open area of the interior of the elongated sleeve 12. The retainer insert 132 has a height Hr that can be any suitable height including, greater than 5 mm, greater than 10 mm, greater than 25 mm, greater than 50 mm and any range between and including the heights provided. As shown in FIG. 13B, the retainer insert has an inner diameter Dr and a thickness Tr. The inner diameter Dr may be configured to substantially match the outer diameter of a medical probe 20. In another exemplary embodiment, the inner diameter of the probe tip retainer is less than that of the medical probe 20, and the probe tip retainer is made of a soft and deformable material allowing the medical probe to be inserted therethrough. The retainer insert shown in FIGS. 13A and 13B for example may be made out of a foam or elastomeric material.

As shown in FIG. 14, an exemplary multifunctional enclosure 10 has a probe tip retainer comprising an indented elongated sleeve portion 134 configured at the near the first end of the elongated sleeve. The indented elongated sleeve portion is a location where the interior diameter of the elongated sleeve is reduced along a portion of the length, and in a preferred embodiment the indented portion is closer to the first end but not along the delicate tip portion of the medical probe 20. The elongated sleeve may comprise a plurality of elongated sleeve portions 134 and 134′ as shown FIG. 14 to reduce stress along the medical probe.

shown in 15, an exemplary multifunctional enclosure 10 has a probe closer retainer 136 type tip retainer 130 that is coupled with the first end closure portion 30. The closure retainer 136 comprises an insert that is configured to fit inside the interior of the elongated sleeve and is attached to the closure portion 30. A closure retainer may be a soft foam sleeve that extends from the closure portion up within the interior of the elongated sleeve. The interior opening of the closure retainer sleeve may be configured to allow for the insertion of the medical probe tip, and protect it from jostling when the multifunctional enclosure is transported.

As shown in 16A and FIG. 16B, an exemplary multifunctional enclosure has a label 140 attached to the elongated sleeve 12. The label shown in FIG. 16A is a second label with the word “Clean” printed thereon. When the second label is removed by pulling on the pull tab 148, or non-adhered portion of the label, a first label 142 may be exposed as shown in FIG. 16B. The second label may be attached to the first label with an adhesive that allows for easy removal without tearing the first label. The first label 142 also has a pull tab 148 for removing the label from the elongated sleeve. Medical personnel may apply a new label comprising a first and second label after the multifunctional enclosure is sterilized or otherwise cleaned. A label as shown in FIGS. 16A and 16B may be an indication label, whereby the status of the multifunctional enclosure and/or the probe configured therein is indicated, such as clean, or dirty. A label may have be configured with an input field, or a place for writing or marking on the label. In another embodiment a label comprises a code, or bar-code for example, that comprises some identifying information about the multifunctional enclosure and/or the medical probe configured therein, such as whether the enclosure is clean or dirty, a tracking code for the probe configured therein, a destination for the probe and/or enclosure and the like.

As shown in 17, an exemplary multifunctional enclosure transport kit 159 comprising a transport cart 150 having wheels 154 and at least one enclosure retainer 152. Four enclosure retainers 152 are configured around an upper retainer support 156, and four enclosure retainers 152′ are configured around a lower retainer support 158. A retainer support is simply a structure that supports the enclosure retainers and may be a platform as shown in FIG. 17 or a hoop, or rod or beam or any combination thereof. FIGS. 18A-18C show side views of a medical probe configured for neurosurgery and attached to a multifunctional enclosure as described herein.

As shown in FIGS. 18A-18C, an exemplary multifunction enclosure 10 is attached to by way of an elastic band 180 and a band retainer opening 182 to a medical probe 20 configured for neurosurgery. The elastic band is attached to the multifunctional enclosure 10 as shown in FIG. 18B and wrapped around a portion of the medical probe 20 and inserted into the band retainer opening 182 configured in the multifunctional enclosure. The multifunctional enclosure 10 comprise a handle housing 260 that extends over a portion of the medical probe. FIG. 18C shows the elastic band secured in the band retainer opening 182. A band retainer opening may be a tapered slit whereby an elastic band and be pinched in the narrowing slit of the opening. The elastic band may securely attach a multifunctional enclosure to a medical probe as shown. The elongated sleeve of the multifunctional enclosure may be rigid and the attachment, as shown, may prevent any damage to the fragile elongated probe member. The elongated sleeve 12 of the multifunction enclosure has a plurality of openings configured therein. A handle housing 260 may be configured to extend completely around the medical probe handle as shown in FIG. 18D and may be configured to be detached from the elongated sleeve portion of the multifunctional enclosure 10. The medical probe handle 262 portion is shown in dashed lines within the handle housing 260 in FIG. 18D

As shown in 19A and 19B, an exemplary label comprising a first label 142 and second label 144 detachably attached together. A label, such as the second label may be configured to be sensitive to sterilization procedures, whereby the label changes color or a message is exposed after being exposed to sterilization. For example, a label may have no message prior to sterilization but may display a message after sterilization, such as CLEAN, or STERILIZED. The label, and the message or background of the message may comprise inks or pigments that are sensitive to heat and/or humidity and/or a specific sterilization gas, such as hydrogen peroxide or ozone for example In another embodiment, a label may comprise inks that change color after exposure to sterilization conditions. FIG. 19B shows the first label 142 after the second label 144 has been peeled off, as shown in FIG. 19A.

As shown in FIG. 20, a person 200 is holding a medical probe 20 having a long and flexible elongated probe member 110 as described herein. Some medical probes may have elongated probe members that are three to five feet long. Handling of these long elongated probe members during procedures can be difficult. A doctor may grasp a portion of the elongated probe member near the insertion point and guide the probe in. The remaining portion of the probe is left unsupported is difficult to manage.

As shown in FIG. 21, an exemplary inflatable support sleeve 210 has a inflation port 212 for inflating the inflatable sleeve. An elongated probe member 110 may be inserted into the inner opening 216 of the inflatable support sleeve 210 and guided therethrough, as shown in FIG. 21B. A release valve 213 may be used to partially deflate the inflatable support sleeve as the elongated probe member 110 is inserted into a patient. In this way, the elongated probe member may be supported throughout the procedure. The inflatable sleeve may be inflated as the elongated probe member is removed from a patient as well.

As shown in FIGS. 22A and 228, an exemplary multifunctional enclosure 10 has a detachable sleeve portion 222. The detachable sleeve portion 222 may be detached, such as by sliding out, as shown in FIGS. 22A and 22B, to provide a support sleeve 220. The support sleeve may be used to support the elongated probe member of a medical probe during a procedure. The detachable sleeve portion 222 may extend along any suitable length SPL of the elongated sleeve 12.

As shown in FIGS. 23A and 23B, an exemplary multifunctional enclosure 10 has a detachable elongated sleeve 12 having a detachable sleeve portion 222. A detachable elongated sleeve 12 having a detachable sleeve portion that extends the entire length of the detachable elongated sleeve allow an elongated medical probe to supported along the entire length, SPL, as shown in FIGS. 24A and B. A stand 240 may further be configured to support a detachable sleeve portion 222 or an inflatable support sleeve, to provide support of the elongated probe member 110 during the insertion and removal. A detachable elongated sleeve 220 may comprise any suitable attachment mechanism, such as an interference fit, a threaded portion, an locking pin and slot configuration and the like.

As shown in FIG. 25, a multifunctional enclosure 10 has a sleeve liner 250 extending down into the elongated sleeve 12. A sleeve liner 250 may be used to prevent contamination of the interior of the elongated sleeve from a probe being reinserted after a procedure. The sleeve liner may be attached to the multifunctional enclosure in any suitable way. A detachable elongated sleeve, may provide for quick and easy installation of a sleeve liner over the end of an elongated sleeve. A sleeve liner may be disposable and a lower cost option than disposing of an elongated sleeve or the complete multifunctional enclosure, as may be the case in some applications.

As shown in FIG. 26 an exemplary multifunctional enclosure 10 comprising a handle housing 260 configured over a portion of a medical probe handle 262. The handle housing 260 comprises a handle 269 to aid in carrying the medical probe configured in the multifunctional enclosure and a extension attachment 268 configured to retain a probe extension, such as an optical or electrical connector. The extension attachment 268 shown is a clip having an inner diameter opening that is configured to retain the extension attachment 265. A elongated sleeve liner 250 is shown disposed inside of the elongated sleeve 12. The elongated sleeve may be detached from the handle housing portion 260 and a liner may be configured over the second end 18 of the elongated sleeve. The sleeve may be deployed, or extended down along the length of the elongated sleeve by applying vacuum to the liner port 264. The handle housing may comprise one or more portions that may be snapped, or otherwise attached around a portion of a medical probe handle.

As shown in FIG. 27 an exemplary telescoping multifunctional enclosure 10 comprises a telescoping elongated sleeve 270. The telescoping elongated sleeve comprises a plurality of telescoping portions, whereby a first telescoping portion 274 is configured at least partially within a second telescoping portion 272. A user may extend one or more telescoping portions and lock connecting segments or portions to provide a set length. The telescoping portions may snap into a retracted configuration and snap into an extended configuration whereby a substantial force is required to alter either configuration. For example, a person may choose to pull on one telescoping portion and may be required to pull substantially hard to overcome the retracted configuration and then extend the telescoping portion to the extended configuration. To lock the telescoping portion in the extended configuration a person may have to pull substantially hard. Any suitable method of configuring telescoping portions may be used.

As shown in FIG. 28, an exemplary funnel shaped elongated sleeve 280 of a multifunctional enclosure 10 has a smaller size at a first end 16 than at a second elongated sleeve end 288. The inside diameter 282 nearer the second end is larger than the inside diameter 283 proximate the extended first end 16.

As shown in FIG. 29A, an exemplary multifunctional enclosure 10 has a bendable segment 160 configured between a first rigid segment 162 and a second rigid segment 163. The multifunctional enclosure is configured in straight configuration for insertion of a medical probe. A medical probe may be inserted into the multifunctional enclosure and the tip of the medical probe may be restrained in the retainer insert 132 configured at the extended end of the elongated sleeve 12. A protective feature 120 is configured around the extended end of the elongated sleeve to further protect the medical probe tip from damage. The bendable segment 160 of the elongated sleeve 12 may comprise any suitable material that will enable the elongated sleeve to be bent without damage to the sleeve. As shown in FIG. 29A, the bendable segment is a corrugated material that may simply be a corrugated portion of the rigid segments of the elongated sleeve, or may comprise a separate segment that is attached to a first and second rigid section. The length FSL of the bendable segment 160 may be any portion of the elongated sleeve length including, but not limited to, no more than about three-quarters, no more than about one-half, no more than about one-third, no more than about one quarter and any range between and including the bendable segment lengths provided. In an exemplary embodiment, as shown in FIG. 29A, a bendable segment is configured between a first and second rigid segments but may be configured after a first rigid segment and extend substantially to the elongated end of the elongated sleeve, or be configured before a second rigid segment and extend substantially to the enlarged end 14.

As shown in FIG. 29B, an exemplary multifunctional enclosure 10 has a bendable segment 160 configured between a first rigid segment 162 and a second rigid segment 163. The multifunctional enclosure is configured in a bent orientation, wherein the elongated sleeve has been bent 180 degrees. A bendable segment may be configured to enable a bend of 180 degrees or more, or 90 degrees or more, or 120 degrees or more without damaging the elongated sleeve or kinking. If the elongated sleeve kinks while being bent, it could damage a medical probe configured therein. A bent retainer 190 is coupled to the elongated sleeve to hold and retain the elongated sleeve in a bent configuration. The first capture portion 192 is coupled to the first rigid segment and the second capture portion 194 is coupled to the second rigid segment. The extension 196 extends between the first and second rigid portions and holds the elongated sleeve in a 180 degree bend configuration. This bent configuration of the elongated sleeve provides more compact storage and transport of the elongated sleeve.

As shown in FIG. 29C, an exemplary bent retainer 190 is configured to retain an elongated sleeve in a bent configuration, as shown attached to the elongated sleeve in FIG. 29B. The exemplary bent retainer comprises a first capture portion 192, a second capture portion 194 and an extension portion 196. The capture portions comprise an opening for insertion of the elongated sleeve. The bent retainer, or at least the capture portions of the bent retainer may be made out of a material that flexes to enable insertion of an elongated sleeve tube, for example.

The multifunctional enclosure as described herein may be used in a variety of ways. In one method of use, a medical probe or the elongated member of a medical probe may be inserted into the multifunctional enclosure as described herein, and the medical probe may be securely transported to and/or from, a medical procedural room, storage area, cleaning facility, repair facility, manufacturer, or a sterilization procedure. In another embodiment, the multifunctional enclosure may be used as a reservoir for fluids that may coated onto the medical probe prior or after a procedure. For example, the medical probe may be coated with a sterilization fluid prior to a procedure by introducing the fluid into the multifunctional enclosure. The medical probe may then be removed from the multifunctional enclosure and inserted into a patient. The fluid may be simply poured into the second end of the multifunctional enclosure, or a filing port 24, may be configured on the multifunctional enclosure for accepting fluid, as shown in FIG. 4. The filling port may have an adjustable, one way, or check valve or a cover to prevent contaminates from entering the multifunctional enclosure. In addition, the medical probe may be coated or treated with a cleaning solution or sterilization fluid after a procedure. For example a cleaning fluid, such as enzyme soap, for example, may be introduced into the multifunctional enclosure, and the medical probe may be inserted into the multifunctional enclosure for post procedural cleaning. Quickly treating the medical probe with a cleaning or sterilization fluid after a procedure may reduce the risk of cross contamination occurring and may provide for more effective and complete removal of biological material.

The multifunctional enclosure as described herein may be a disposable, or may be reusable. A first multifunctional enclosure may be used to sterilization and transport to a medical procedure and a second multifunctional enclosure may be used for post treatment transport and cleaning.

### Example

A 30 in long polyethylene terephthalate (PETG) tube having an outer diameter of 0.848 in and a wall thickness of 0.022 in was flared at one end. The flared end, or second end, had a funnel shape and a second end diameter of 1.8 in, and the flared length was approximately 1 in, as measured along the length axis of device. A latex free, synthetic rubber elastic band approximately 3 in in diameter 0.125 in wide and 0.0625 was configured around the elongated sleeve. A sterilization enclosure pouch available from AMCOR Ltd., having a paper side and a plastic side was attached to the multifunctional enclosure, by a combination of heat sealing and a double sided tape.

### DEFINITIONS

Multifunctional enclosure as used herein is defined as an enclosure that is configured to cover at least the elongated portion of a medical probe and is not configured for insertion into the body.

Medical probe as used herein is defined as any medical device that is inserted into the body for the purpose of imaging, diagnosis and treatment, including but not limited to endoscope, catheter, pressure transducer or ultrasonic imaging device. A medical probe may comprise a flexible or rigid elongated member.

Rigid as used herein in reference to the elongated sleeve portion, or rigid segment of the multifunctional enclosure, means that the elongated sleeve will prevent the elongated member of the medical probe from swinging or moving freely. The elongated sleeve may have some flexible properties, such as a plastic tube that may bend to a certain degree under a load, but will permanently deform if bent too far, such as more than about 90 degrees, for example.

The term detachably attached, as used herein in reference to the first end closure portion, means that the first end closure portion can be temporarily attached to the first end of the multifunctional enclosure, such as by screwing, pushing, or snapping on, for example.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

I claim:

1. A multifunctional enclosure for medical probes comprising:
a. an elongated sleeve comprising:
i. a first end; and
ii. a second end;
wherein the second end is configured with an enlarged opening for the insertion of an elongated member of a medical probe.

2. A multifunctional enclosure for medical probes of claim 1 comprising:
a. a protective feature configured on a portion of the first end; and
wherein the second end is configured with an enlarged opening for the insertion of an elongated member of a medical probe.

3. The multifunctional enclosure of claim 2 wherein the protective feature covers the first end and extends up the elongated sleeve at least 50 mm.

4. A multifunctional enclosure for medical probes of claim 1 comprising a handle housing configured to fit over a handle portion of a medical probe.

5. The multifunctional enclosure for medical probes of claim 4, wherein the handle housing comprises:
a. band; and
b. a band retainer opening that is tapered;
whereby said band is configured to be retained in a narrowed portion of the band retainer opening.

6. The multifunctional enclosure for medical probes of claim 4, wherein the handle housing is configured to fit substantially over the entire handle of a medical probe.

7. The multifunctional enclosure for medical probes of claim 4, wherein the handle housing is configured to be detachable from said multifunctional enclosure.

8. The multifunctional enclosure for medical probes of claim 1, wherein the elongated sleeve comprises:
a. a funnel shaped elongated sleeve portion having:
i. a first inside diameter proximate the first end;
ii. a second inside diameter proximate the second end;
wherein said first inside diameter is smaller than said second inside diameter.

9. A multifunctional enclosure for medical probes of claim 1, wherein the elongated sleeve comprises:
a. an telescoping elongated sleeve portion comprising:
i. a plurality of telescoping portions.

10. A multifunctional enclosure for medical probes of claim 9 wherein the telescoping elongated sleeve comprises
i. a first telescoping portion;
ii. a second telescoping portion;
wherein the first telescoping portion is configured at least partially within said second telescoping portion.

11. A multifunctional enclosure for medical probes of claim 1 comprising a bendable segment configured along the elongated sleeve.

12. A multifunctional enclosure for medical probes of claim 11, wherein the bendable segment is configured between a first rigid segment and a second rigid segment.

13. A multifunctional enclosure for medical probes of claim 11, wherein the bendable segment comprises a corrugated material.

14. A multifunctional enclosure for medical probes of claim 11, further comprising a bent retainer configured to retain the elongated sleeve in a bent configuration.

15. A multifunctional enclosure for medical probes comprising:
a. an elongated sleeve comprising:
i. a first end;
ii. a second end; and
iii. a length;
wherein the second end is configured with an enlarged opening for the insertion of an elongated member of a medical probe an elongated sleeve having a first end and a second end, and
b. a detachable elongated sleeve liner;
wherein the sleeve liner is configured to extend within a portion of the elongated sleeve, whereby said elongated member of said medical probe is configured for retention within said elongated sleeve liner within said elongated sleeve.

16. The multifunctional enclosure for medical probes of claim 15, wherein the elongated sleeve liner is configured to extend substantially along the entire length of said elongated sleeve.

17. The multifunctional enclosure for medical probes of claim 15, wherein the elongated sleeve comprises a liner port.

18. The multifunctional enclosure for medical probes of claim 15, wherein the elongated sleeve comprises a liner port configured proximate the first end of said elongated sleeve, whereby vacuum can be used to extend the elongated sleeve liner into said elongated sleeve.

19. The multifunctional enclosure for medical probes of claim 15, wherein the elongated sleeve is detachable from the multifunctional enclosure.
